(19) United States
(12) Patent Application Publication
(10) Pub. No.: US 2024/0226041 A1
(43) Pub. Date: Jul. 11, 2024

(54) METHODS AND COMPOSITIONS FOR TREATMENT OF NEUROPATHIC PAIN USING STAT3 INHIBITORS

(71) Applicant: BOARD OF REGENTS, THE UNIVERSITY OF TEXAS SYSTEM, Austin, TX (US)

(72) Inventors: David TWEARDY, Houston, TX (US); Moses Makokha KASEMBELI, Houston, TX (US); Anniemieke KAVELAARS, Houston, TX (US); Cobi Jacoba Johanna HEIJNEN, Houston, TX (US)

(73) Assignee: BOARD OF REGENTS, THE UNIVERSITY OF TEXAS SYSTEM, Austin, TX (US)

(21) Appl. No.: 18/559,025

(22) PCT Filed: May 5, 2022
(86) PCT No.: PCT/US2022/027869

§ 371 Date: Nov. 3, 2023

Related U.S. Application Data
(60) Provisional application No. 63/184,572, filed on May 5, 2021.

(51) Int. Cl. A61K 31/18; A61K 33/243; A61K 45/06; A61P 25/02

(52) U.S. Cl. CPC A61K 31/18; A61K 33/243; A61K 45/06; A61P 25/02

(57) ABSTRACT

Disclosed herein are methods and compositions for treatment and prevention of neuropathic pain conditions. Aspects of the present disclosure are directed to methods for treatment of neuropathic pain conditions using STAT3 inhibitors. Certain aspects pertain to TTI-101 and methods for use in treatment of chemotherapy-induced peripheral neuropathy, mechanical nerve injury, and other neuropathic pain conditions. Also disclosed are methods for treatment of chemotherapy-induced peripheral neuropathy comprising administering TTI-101 and one or more chemotherapeutic agents to a subject with cancer.

This application claims priority of U.S. Provisional Patent Application No. 63/184,572, filed May 5, 2021, which is hereby incorporated by reference in its entirety.

### SEQUENCE LISTING

The instant application contains a Sequence Listing which has been submitted in ASCII format and is hereby incorporated by reference in its entirety. Said ASCII copy, created on May 4, 2022, is named MDAC_P1263WO_Sequence_Listing.txt and is 5,506 bytes in size.

### BACKGROUND

### I. Field of the Invention

Aspects of this invention relate to at least the fields of molecular biology, biochemistry, and medicine. Particular aspects relate to STAT3 inhibitors for treatment of neuropathic pain conditions, including chemotherapy-induced peripheral neuropathy and traumatic nerve injury.

### II. Background

Signal Transducer and Activator of Transcription 3 (STAT3) is known to play an essential role in biological processes important for development, including cell growth and survival, as well as in restoring homeostasis after injury. However, persistent STAT3 signaling has been linked to a number of pathological conditions including cancer, chronic inflammation, and fibrosis. An extensive body of preclinical data indicates that inhibition of STAT3 activity may be of substantial therapeutic benefit (Bharadwaj, Kasembeli, Robinson & Tweardy, 2020; Kasembeli, Bharadwaj. Robinson & Tweardy, 2018). However, agents that target STAT3 have been slow to enter the clinic, in part, because of difficulties inherent in targeting transcription factors, a class of proteins deemed “undruggable” due to the large size of their protein-protein interaction interfaces (Dang. Reddy, Shokat & Soucek, 2017). In addition, serious adverse events (SAE), including lactic acidosis and peripheral neuropathy, have been observed with some small-molecule STAT3 inhibitors in clinical-stage development (Bendell et al., 2014; Ogura et al., 2015). These have been attributed to targeting of STAT3's non-canonical functions, most notably, its contribution to mitochondrial-mediated oxidative phosphorylation (Genini et al., 2017), which relies on phosphorylation of STAT3 on serine 727, in contrast to phosphorylation on tyrosine 705 required for its canonical function (Garama et al., 2015; Yang & Rincon, 2016).

Many STAT3-directed drug development programs have focused on STAT3's SH2 domain, in particular its phosphotyrosine (pY) peptide binding pocket. However, the finding that some inhibitors induce mitochondrial toxicity suggests they may target other regions of STAT3 and affect STAT3 structure and stability. In fact, Genini et al. demonstrated that OPB-51602, and other small-molecule STAT3 inhibitors designed to directly target STAT3, caused STAT3 aggregation and altered intracellular protein homeostasis (Genini et al., 2017). They further argued that induction of cell death by these agents is mediated, in part, through a proteotoxic mechanism in metabolically stressed cancer cells and suggested that this may be a common mechanism underlying the anticancer activity of any inhibitor that directly targets the SH2 domain within STAT3.

Common neuropathic pain conditions include posttraumatic neuropathic pain, chemotherapy-induced peripheral neuropathy (CIPN), postherpetic neuralgia, central pain, cancer neuropathic pain, phantom pain, complex regional pain syndrome, radiculopathy, and failed back surgery syndrome. Posttraumatic neuropathic pain occurs in up to half or more of patients who suffer traumatic nerve or spinal cord injury. Current therapies fail in many patients. Chemotherapy-induced peripheral neuropathy (CIPN) affects up to 80% of cancer patients treated with chemotherapy agents and is characterized by progressive and irreversible damage to the peripheral nervous system. Treatment of CIPN is mainly symptomatic and remains a significant clinical challenge. A greater understanding of the mechanisms behind neuropathic pain are required in order to develop more effective therapies.

There exists a need for compositions and methods for effective treatment and prevention of CIPN, traumatic nerve injury, and other neuropathic pain conditions.

### SUMMARY

Aspects of the present disclosure provide methods and compositions for treatment and prevention of neuropathic pain conditions, including chemotherapy-induced peripheral neuropathy (CIPN) and mechanical nerve injury, using STAT3 inhibitors such as TTI-101. Accordingly, disclosed are methods for treatment of CIPN comprising administering to a subject a therapeutically effective amount of TTI-101 or a pharmaceutically acceptable salt thereof. Also disclosed are methods for treatment of mechanical nerve injury comprising administering to a subject a therapeutically effective amount of TTI-101 or a pharmaceutically acceptable salt thereof. Aspects of the disclosure comprise complete alleviation of one or more symptoms of mechanical nerve injury by administering TTI-101 or a pharmaceutically acceptable salt thereof.

Embodiments of the present disclosure include methods for treatment of a neuropathic pain condition, methods for treatment of chemotherapy-induced peripheral neuropathy, methods for treatment of mechanical nerve injury, methods for treatment of traumatic nerve injury, methods for complete alleviation of one or more symptoms of a neuropathic pain condition, and methods for simultaneous treatment of cancer and chemotherapy-induced peripheral neuropathy. The disclosed methods can include at least 1, 2, 3, or more of the following steps: diagnosing a subject as having a neuropathic pain condition, diagnosing a subject as having chemotherapy-induced peripheral neuropathy, diagnosing a subject as having mechanical nerve injury, diagnosing a subject as having cancer, measuring mitochondrial function in a subject, detecting lactic acidosis in a subject, detecting allodynia in a subject, detecting hyperalgesia in a subject, administering a therapeutically effective amount of a pharmaceutical composition comprising TTI-101, and administering a therapeutically effective amount of a pharmaceutical composition comprising a pharmaceutically acceptable salt of TTI-101. Any one or more of the preceding steps may be excluded from embodiments of the disclosure.

Disclosed herein, in some embodiments, is a method of treating a subject for mechanical nerve injury, the method comprising administering to the subject a therapeutically effective amount of a pharmaceutical composition comprising TTI-101 or a pharmaceutically acceptable salt thereof. In some embodiments, the subject does not have cancer. In some embodiments, the subject is not suspected of having cancer. In some embodiments, the mechanical nerve injury is traumatic nerve injury. In some embodiments, the mechanical nerve injury is carpal tunnel syndrome. In some embodiments, the mechanical nerve injury is vertebral disk herniation. In some embodiments, administering the pharmaceutical composition to the subject completely alleviates one or more symptoms of the mechanical nerve injury in the subject. In some embodiments, the one or more symptoms comprise allodynia. In some embodiments, the one or more symptoms comprise hyperalgesia. In some embodiments, the pharmaceutical composition is not administered to the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, or 200 days (or any range or value derivable therein), or more, following complete alleviation of the one or more symptoms. In some embodiments, the pharmaceutical composition is not administered to the subject for at least 50 days following complete alleviation of the one or more symptoms. In some embodiments, the method further comprises administering to the subject an additional therapeutic agent. In some embodiments, the additional therapeutic agent is an analgesic. In some embodiments, the analgesic is an opioid, a nonsteroidal anti-inflammatory drug (NSAID), acetaminophen, a steroid, a COX-2 inhibitor, a topical analgesic, or any combination thereof. In some embodiments, the subject was previously treated for the mechanical nerve injury with a previous treatment. In some embodiments, the subject was determined to be resistant to the previous treatment.

In some embodiments, the pharmaceutical composition is administered to the subject orally. In some embodiments, the TTI-101 or pharmaceutically acceptable salt thereof is administered at a concentration of at least, at most, or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, or 30 mg/kg, or any range or value derivable therein. In some embodiments, the TTI-101 or pharmaceutically acceptable salt thereof is administered at a concentration of at most 15 mg/kg. In some embodiments, the TTI-101 or pharmaceutically acceptable salt thereof is administered at a concentration of at most 5 mg/kg. In some embodiments, the TTI-101 or pharmaceutically acceptable salt thereof is administered at a concentration of at most 1 mg/kg. In some embodiments, mitochondrial function of the subject following administration of the pharmaceutical composition is not reduced relative to mitochondrial function of the subject prior to administration of the pharmaceutical composition. In some embodiments, the pharmaceutical composition is administered to the subject once per day for multiple days. In some embodiments, the pharmaceutical composition is administered to the subject every other day for multiple days.

Also disclosed herein, in some embodiments, is a method of treating a subject for chemotherapy-induced peripheral neuropathy, the method comprising administering to the subject a therapeutically effective amount of a pharmaceutical composition comprising TTI-101 or a pharmaceutically acceptable salt thereof. In some embodiments, administering the pharmaceutical composition to the subject completely alleviates a symptom of the chemotherapy-induced peripheral neuropathy in the subject. In some embodiments, the symptom is allodynia. In some embodiments, the symptom is hyperalgesia. In some embodiments, the subject was previously treated with a chemotherapeutic. In some embodiments, the chemotherapeutic is a platinum-containing chemotherapeutic. In some embodiments, the chemotherapeutic is cisplatin. In some embodiments, the chemotherapeutic is oxaliplatin. In some embodiments, the chemotherapeutic is carboplatin. In some embodiments, the subject was previously treated for chemotherapy-induced peripheral neuropathy with a previous treatment. In some embodiments, the subject was determined to be resistant to the previous treatment.

In some embodiments, the method further comprises administering to the subject a chemotherapeutic. In some embodiments, the pharmaceutical composition comprises the chemotherapeutic. In some embodiments, the pharmaceutical composition does not comprise the chemotherapeutic. In some embodiments, the pharmaceutical composition and the chemotherapeutic are administered substantially simultaneously. In some embodiments, the pharmaceutical composition and the chemotherapeutic are administered sequentially. In some embodiments, the pharmaceutical composition is administered prior to administering the chemotherapeutic. In some embodiments, the pharmaceutical composition is administered subsequent to administering the chemotherapeutic. In some embodiments, the chemotherapeutic is a platinum-containing chemotherapeutic. In some embodiments, the chemotherapeutic is cisplatin. In some embodiments, the chemotherapeutic is oxaliplatin. In some embodiments, the chemotherapeutic is carboplatin.

Also disclosed herein, in some embodiments, is a method of treating a subject for a neuropathic pain condition, the method comprising administering to the subject a therapeutically effective amount of a pharmaceutical composition comprising TTI-101 or a pharmaceutically acceptable salt thereof. In some embodiments, the neuropathic pain condition is mechanical nerve injury. In some embodiments, the neuropathic pain condition is mechanical nerve injury, a metabolic disease, a neurotropic viral disease, an inflammatory condition, nervous system focal ischemia, or a combination thereof. In some embodiments, the neuropathic pain condition is chemotherapy-induced peripheral neuropathy. Any one or more of these conditions are contemplated herein. One or more of these conditions may be excluded from certain embodiments of the disclosure.

Throughout this application, the term “about” is used to indicate that a value includes the inherent variation of error for the measurement or quantitation method.

“Individual, “subject,” and “patient” are used interchangeably and can refer to a human or non-human.

The use of the word “a” or “an” when used in conjunction with the term “comprising” may mean “one,” but it is also consistent with the meaning of “one or more.” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of.” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Examples section are understood to be embodiments that are applicable to all aspects of the technology described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C show the effects of STAT3 inhibitors on mitochondrial function. DU-145 cells were treated with the indicated STAT3 inhibitors at 30 μM for 2 Hrs. FIG. 1A shows seahorse experiments showing oxygen consumption rate (OCR) by DU-145 cells treated with DMSO alone or DMSO containing the indicated STAT3 inhibitor at 30 μM prior to and following addition of oligomycin, FCCP, and antimycin A/rotenone, as indicated (n=6). FIG. 1B shows the results shown in FIG. 1A combined into one graph, showing all the OCR curves relative to each other. FIG. 1C shows mean±SEM of basal respiration, maximal respiration, spare respiratory capacity, and ATP production as determine from the six independent Seahorse assays (n=6).

FIG. 2 shows immunoblotting of fractions of DU-145 cells incubated with indicated drugs (10 μM concentration for 16 Hrs). Fractions were separated using 4-20% SDS-PAGE and immunoblotted using antibodies against STAT3, Histone H2B, GAPDH and vimentin. Data are representative of three independently performed experiments.

FIG. 3 shows stability of compounds incubated with GSH. The AUC of each compound measured at the times indicated by UV-HPLC and expressed as percent of the starting AUC of the peaks. Data are representative of four independently performed experiments.

FIGS. 4A-4C show alkylation of STAT3 by Stattic. FIG. 4A shows a schematic depicting chemistry of possible alkylation of STAT3 by Stattic and results of LC-MS chromatograms of STAT3 peptides of alkylated peptides, as predicted from reaction chemistry. FIG. 4B shows results of LC-MS/MS demonstrating covalent modification of STAT3 by Stattic. Chromatograms show fragment ion analysis revealing alkylation of each cysteine-containing peptide, as indicated. Mass Spectra were annotated using IPSA. Representative data of four independent experiments. FIG. 4C shows the amino acid sequence of STAT3βtr indicating cysteine residues modified. Red residues indicates cysteine-containing tryptic fragments identified by LC-MS/MS, with some peptides containing more than one modified cysteine. Bolded residues are within tryptic fragments that can be identified by LC-MS/MS. Residues that are not bolded are within tryptic fragments that are either too large or too small to be detected.

FIG. 5 shows results of alkylation studies of STAT3 by TTI-101. Shown on the left is a schematic depicting the chemistry of possible alkylation of STAT3 by TTI-101; shown on the right are the LC-MS chromatograms of STAT3 peptides, showing no MRM signal for predicted peptide adducts, indicating that no peptides were alkylated.

FIGS. 6A-6B show results demonstrating that TTI-101 does not cause mechanical allodynia, but rather reverses mechanical allodynia caused by cisplatin. FIG. 6A shows results from studies in which male C57/B16 mice (n=8 per group) received TTI-101 (50 mg/kg i.p. every other day) and mechanical allodynia was assessed using von Frey hairs and the up-and-down method. FIG. 6B shows results from studies in which male C57/B16 mice (n=4 per group) were treated with cisplatin (two rounds of 5 daily doses of 2.3 mg/kg i.p. followed by 5 days of rest). Dosing with TTI-101 (50 mg/kg i.p. every other day) started 12 days after the last dose of cisplatin. Data were analyzed by two-way ANOVA repeated measures. Time: P<0.001; Group: P<0.001; Interaction P<0.001. **P<0.01 Tukey multiple comparison test. Data are from a representative experiment out of two with similar results.

FIGS. 7A-7B show results demonstrating that TTI-101 reverses SNI-induced allodynia. Male and female mice underwent SNI surgery and were treated with TTI-101 (Males: n=5; females: n=6) or vehicle (Males: n=4; Females: n=6) by oral gavage for 6 doses every other day from day 10 after SNI. FIG. 7A shows mechanical allodynia in male and female mice. Data are shown as mean±SEM and were analyzed using two-way ANOVA followed by Sidak's post-hoc test. *P<0.05. FIG. 7B shows results demonstrating that no signs of mechanical allodynia at 31 and 52 days after start of TTI-1010 treatment (19 and 40 days after the last dose). Data are from 4 vehicle-treated and 5 TTI-101-treated male mice per group). Two-way ANOVA followed by Sidak's post-hoc test: P<0.05.

FIGS. 8A-8C show results demonstrating the effect of TTI-101 on the DRG transcriptome of cisplatin-treated mice. FIG. 8A shows genes differentially expressed among groups is shown in a Venn diagram. Expression of 1,973 genes was changed in response to cisplatin when compared to the PBS mice (PBS vs. Cis; n=3 male mice per group). Expression of 1,713 genes was changed in response to TTI-101 administration vs. mice treated with cisplatin alone (Cis vs. Cis+TTI-101). A cutoff of (−0.2<log 2 Fold Change <0.2) and p=0.1 was used for the analysis. Expression of 2,154 genes was changes in response to TTI-101 administration compared to PBS mice (PBS vs TTI-101). FIG. 8B shows subcellular clustering of 443 overlapping genes showing directionality of expression. Up-regulated and downregulated genes are highlighted in red and green, respectively. Gray indicates effect cannot be predicted. FIG. 8C shows the top IPA canonical pathways along with −log (p-value) assigned to 443 common genes between PBS vs. Cis and Cis vs. Cis+TTI-101 mice.

FIGS. 9A-9B show the results of IPA comparison analyses described in Example 1. FIG. 9A shows the top upstream regulators driving TTI-101-dependent changes in DRG identified using the IPA comparison analysis tool. IPA core analysis was performed between PBS vs. Cisplatin and Cisplatin vs. Cisplatin+TTI-101, followed by comparison of the two core analyses. FIG. 9B shows a heat map showing details of target genes in VEGF network. Fold change data for target genes upregulated (blue) or downregulated (red) is shown

FIGS. 10A-10C show results demonstrating the effects of STAT3 inhibitors on mitochondrial functions. DU-145 cells were treated with STAT3 inhibitors at 10-30 μM for 2 Hrs. Data are representative of 4 or more independent experiments. FIG. 10A shows Seahorse experiment measuring the OCR for DU-145 cells treated with STAT3 inhibitor or DMSO control prior to addition of oligomycin, FCCP, and antimycin A/rotenone. FIG. 10B shows mean±SEM OCR indicative of basal respiration, maximal respiration, spare respiratory capacity and ATP production determined from Seahorse assays after treatment of cells with the indicated inhibitors (n=4). FIG. 10C shows mean±SEM OCR indicative of the coupling efficiency and proton leak determined from Seahorse assays after treatment of cells with inhibitors 30 μM concentration.

FIGS. 11A-11C show alkylation of STAT3βtr by iodoacetamide and NEM. FIGS. 11A-11B show a schematic depicting chemistry of possible alkylation of STAT3 by iodoacetamide (FIG. 11A) and NEM (FIG. 11B) and the results of LC-MS chromatograms of STAT3 peptides demonstrating alkylated peptides, as predicted from the chemistry. FIG. 11C shows Z-score histograms comparing mass shifts of STAT3 peptides incubated with STATTIC, TTI101, or TTI101ox vs. STAT3 incubated with DMSO. The dotted line indicates the cutoff for a significant Z-score. The peptide mass peak shifted by 211 Da represents the predicted addition of STATTIC as a chemical adduct; no significant mass shifts were observed with TTI-101 or with TTI 101ox indicating that neither forms chemical adducts with STAT3.

FIG. 12 shows results from an additional experiment examining effect of TTI-101 on cisplatin-induced mechanical allodynia. Male C57/B16 mice (n=4 per group) were treated with cisplatin (two rounds of 5 daily doses of 2.3 mg/kg i.p. followed by 5 days of rest). Dosing with TTI-101 (50 mg/kg i.p. every other day) started 13 days after the last dose of cisplatin. Data were analyzed by two-way ANOVA repeated measures. Time: P<0.001; Group: P<0.001; Interaction P<0.01. **P<0.01 Tukey multiple comparison test.

FIG. 13 shows the mechanistic networks of four upstream regulators involving STAT3 as an intermediate regulator. Note that STAT3 (black outline) is an intermediate regulator in all networks and it is predicted to be inhibited. Color orange and blue indicate activation or inhibition respectively. Yellow arrow represents inconsistent relationship when the expected direction is different from direction observed.

FIGS. 14A-14C show results demonstrating that TTI-101 reduces inflammation, oxidative stress and barrier permeability in spared nerve injury (SNI) mice. FIG. 14A shows genes upregulated by TTI-101. FIG. 14B shows genes downregulated by TTI-101. FIG. 14C shows a volcano plot of genes significantly downregulated and upregulated by TTI-101.

### DETAILED DESCRIPTION

TTI-101 (also C188-9), is a competitive inhibitor of STAT3 designed to target the pY-peptide binding site within STAT3's SH2 domain and thereby directly block two key steps in its activation-recruitment to activated cytokine receptor complexes and homodimerization (Bharadwaj et al., 2016; Bharadwaj, Kasembeli & Tweardy, 2016). Good laboratory practice (GLP)-compliant, 28-day pharmacotoxicology studies have been performed of TTI-101 (Bharadwaj, Kasembeli & Tweardy, 2016), which demonstrated no drug-related toxicity up to the maximum dose administered.

Aspects of the present disclosure are based, at least in part, on the surprising discovery that TTI-101 does not affect mitochondrial function, chemically modify STAT3, cause STAT3 aggregation in metabolically stressed cells, or cause peripheral neuropathy; instead TTI-101 administration unexpectedly reversed mechanical allodynia in models of chemotherapy-induced peripheral neuropathy (CIPN) and spared nerve injury (SNI). As disclosed herein, TTI-101 is useful in treatment of nerve injury conditions including CIPN and mechanical nerve injury. In certain aspects, TTI-101 may be of special benefit when administered to patients receiving CIPN-inducing agents, such as platinum-based chemotherapeutics, as part of their cancer therapy regimen.

### I. Neuropathic Pain Conditions

Aspects of the present disclosure comprise methods for treatment or prevention of neuropathic pain conditions. As used herein, a “neuropathic pain condition” describes any disease, disorder, or other condition that is characterized by the presence of neuropathic pain. Neuropathic pain describes any pain caused by an injury, disease, or disorder of the nervous system. Various neuropathic pain conditions are known in the art and contemplated herein, examples of which are provided in Zimmermann M. Pathobiology of neuropathic pain. Eur J Pharmacol. 2001 Oct. 19; 429 (1-3):23-37 and Zilliox L A. Neuropathic Pain. Continuum (Minneap Minn). 2017 Apr.; 23(2, Selected Topics in Outpatient Neurology): 512-532, each of which is incorporated herein by reference in its entirety.

Aspects of the present disclosure are directed to methods for treatment of a neuropathic pain condition in a subject comprising administering an effective amount of TTI-101, a derivative thereof, or a pharmaceutically acceptable salt thereof. In some embodiments, disclosed are methods for treatment of neuropathic pain comprising administering an effective amount of TTI-101. Non-limiting examples of neuropathic pain conditions which may be treated using compositions of the present disclosure (e.g., TTI-101) include: mechanical nerve injury (e.g., traumatic nerve injury, carpal tunnel syndrome, vertebral disk herniation), metabolic disease (diabetic polyneuropathy), neurotropic viral disease (e.g., herpes zoster, HIV), neurotoxicity (e.g., chemotherapy-induced peripheral neuropathy, tuberculosis-induced neuropathy), multiple sclerosis, nervous system focal ischemia (e.g., thalamic pain syndrome), postherpetic neuralgia, central pain, cancer neuropathic pain, phantom pain, posttraumatic neuropathic pain, radiculopathy and failed back surgery syndrome, and complex regional pain syndrome.

Certain embodiments pertain to partial or complete alleviation of one or more symptoms of a neuropathic pain condition. In some embodiments, the disclosed methods completely alleviate one or more symptoms of a neuropathic pain condition in a subject. The one or more symptoms may comprise, for example, allodynia, hyperalgesia, or a combination thereof. As disclosed herein, TTI-101 may be used to completely alleviate a symptom of a neuropathic pain disorder (e.g., allodynia, hyperalgesia, etc.), after which administration may be ceased without resurgence of the symptom for an extended period of time. Accordingly, a composition of the disclosure (e.g., TTI-101) may be administered to a subject multiple times until complete alleviation of the one or more symptoms, after which the composition may not be administered to the subject for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, or 200 days (or any range or value derivable therein), or more. In some embodiments, TTI-101 is administered to a subject having a neuropathic pain condition until complete alleviation of one or more symptoms of the condition, after which TTI-101 is not administered to the subject for at least 10 days. In some embodiments, TTI-101 is administered to a subject having a neuropathic pain condition until complete alleviation of one or more symptoms of the condition, after which TTI-101 is not administered to the subject for at least 20 days. In some embodiments, TTI-101 is administered to a subject having a neuropathic pain condition until complete alleviation of one or more symptoms of the condition, after which TTI-101 is not administered to the subject for at least 30 days. In some embodiments, TTI-101 is administered to a subject having a neuropathic pain condition until complete alleviation of one or more symptoms of the condition, after which TTI-101 is not administered to the subject for at least 40 days. In some embodiments, TTI-101 is administered to a subject having a neuropathic pain condition until complete alleviation of one or more symptoms of the condition, after which TTI-101 is not administered to the subject for at least 50 days.

In some embodiments, the neuropathic pain condition is chemotherapy-induced peripheral neuropathy (CIPN). Accordingly, in some embodiments, disclosed is a method for treatment of CIPN comprising administering to a subject an effective amount of TTI-101 or a pharmaceutically acceptable salt thereof. The term “chemotherapy-induced peripheral neuropathy”, describes a condition of neuropathic pain that is induced by a chemotherapeutic agent, including but not limited to a platinum-containing chemotherapeutic such as cisplatin, carboplatin, and oxaliplatin.

In some embodiments, the neuropathic pain condition is mechanical nerve injury (e.g., traumatic nerve injury, carpal tunnel syndrome, vertebral disk herniation, etc.). Accordingly, in some embodiments, disclosed is a method for treatment of mechanical nerve injury comprising administering to a subject an effective amount of TTI-101 or a pharmaceutically acceptable salt thereof.

### II. STAT3 Inhibitors and TTI-101

Aspects of the present disclosure are directed to STAT3 inhibitors and methods of use. In some embodiments, disclosed are STAT3 inhibitors which do not affect STAT3 mitochondrial function, cause STAT3 aggregation, chemically modify STAT3, or induce peripheral neuropathy. Certain aspects of the present disclosure are directed to TTI-101 (also “C188-9” or “Cpd188-9”), and pharmaceutically acceptable salts thereof, as well as methods of use in treatment of neuropathic pain conditions, including CIPN, traumatic nerve injury, and chronic pain. TTI-101 is a compound having formula:

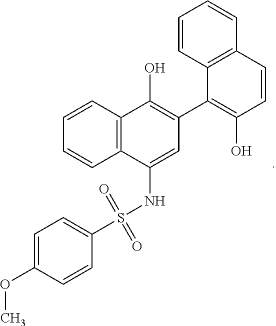 [SMILES: COc1ccc(S(=O)(=O)Nc2cc(-c3c(O)ccc4ccccc34)c(O)c3ccccc23)cc1]

TTI-101 is a STAT3 inhibitor and is described in, for example, U.S. Pat. No. 8,779,001 and Zhang L, et al., Am J Physiol Renal Physiol. 2020 Jul. 1; 319 (1): F84-F92, each incorporated by reference herein in its entirety. Also contemplated herein are derivatives of TTI-101 capable of inhibiting STAT3. TTI-101 and various derivatives of TTI-101 capable of inhibiting STAT3 are described in, for example, U.S. Patent Application Publication 2021/0038544, incorporated herein by reference in its entirety.

Aspects of the present disclosure are directed to pharmaceutical compositions comprising TTI-101 or a pharmaceutically acceptable salt thereof. Additional aspects are directed to methods for treatment of neuropathic pain conditions (e.g., CIPN, traumatic nerve injury, chronic pain, etc.) comprising administering an effective amount of a pharmaceutical composition comprising TTI-101 or a pharmaceutically acceptable salt thereof.

### III. Therapeutic Methods

Aspects of the present disclosure comprise therapeutic methods and compositions for use thereof. Compositions of the disclosure may be used for in vivo, in vitro, and/or ex vivo administration.

### A. Cancer Therapy

In some embodiments, the disclosed methods comprise administering a cancer therapy to a subject or patient. The cancer therapy may be chosen based on an expression level measurements, alone or in combination with the clinical risk score calculated for the subject. The cancer therapy may be chosen based on a genotype of a subject. The cancer therapy may be chosen based on the presence or absence of one or more polymorphisms in a subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is a Stage I cancer. In some embodiments, the cancer is a Stage II cancer. In some embodiments, the cancer is a Stage III cancer. In some embodiments, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; cpendymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia.

In some embodiments, disclosed are methods for treating cancer originating from the prostate. In some embodiments, the cancer is prostate cancer. In some embodiments, the cancer is breast cancer. In some embodiments, the cancer is a recurrent cancer. In some embodiments, the cancer is an immunotherapy-resistant cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

Biomarkers, like SNPs, can, in some cases, predict the efficacy of certain therapeutic regimens and can be used to identify patients who will receive benefit from a particular therapy.

### 1. Radiotherapy

In some embodiments, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of radiotherapy administered to a subject may be presented as a total dose of radiotherapy, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67. 68, 69, 70, 71, 72, 73, 74, 75, 76, 77, 78, 79, 80. 81, 82, 83, 84, 85, 86, 87, 88, 89, 90, 91. 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92. 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### 2. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

a. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy.” or “CBI”), refers to cancer therapy comprising providing one or more immune checkpoint inhibitors to a subject suffering from or suspected of having cancer.

b. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

d. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. Immuno Therapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

e. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another embodiment, the antibody has at least about 70, 75, 80, 85, 90, 95, 97, or 99% (or any derivable range or value therein) variable region amino acid sequence identity with the above-mentioned antibodies.

### 3. Activator of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 4. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3. TLR7. TLR8 or CD40 have been used as antibody targets.

### 5. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 6. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 7. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein.

### 8. Chemotherapies

In some embodiments, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidin analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum-containing chemotherapeutic agents (e.g., cisplatin, carboplatin, oxalipaltin, nedaplatin, triplatin tetranitrate, phenanthriplatin, picoplatin, and satraplatin), substituted ureas (e.g., hydroxyurca), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### B. Therapeutic Combinations

The therapy provided herein may comprise administration of a combination of therapeutic agents, such as TTI-101 and a chemotherapeutic. The therapies may be administered in any suitable manner known in the art. For example, TTI-101 and the chemotherapeutic may be administered sequentially (at different times) or concurrently (at approximately the same time; also “substantially simultaneously”). In some embodiments, TTI-101 and the chemotherapeutic are administered in a separate composition. In some embodiments, TTI-101 and the chemotherapeutic are in the same composition.

In some embodiments, TTI-101 and the chemotherapeutic are administered substantially simultaneously. In some embodiments, TTI-101 and the chemotherapeutic are administered sequentially. In some embodiments, TTI-101, the chemotherapeutic, and an additional therapeutic (e.g., an immunotherapeutic) are administered sequentially. In some embodiments, TTI-101 is administered before administering the chemotherapeutic. In some embodiments, TTI-101 is administered after administering the chemotherapeutic.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician. In some embodiments, the disclosed methods comprise oral administration of TTI-101 to a subject.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, TTI-101 is administered to a subject (with or without an additional agent such as a chemotherapeutic) at a dose of between 1 mg/kg and 5000 mg/kg. In some embodiments, TTI-101 is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range or value derivable therein. In some embodiments, TTI-101 is administered to a subject at a dose of at most 50, 45, 40, 35, 30, 25, 20, 15, 10, 5, 2.5, or 1 mg/kg, or less. In some embodiments, TTI-101 is administered to a subject at a dose of at most 15 mg/kg. In some embodiments, TTI-101 is administered to a subject at a dose of at most 5 mg/kg. In some embodiments, TTI-101 is administered to a subject at a dose of at most 1 mg/kg.

In some embodiments, a single dose of a chemotherapeutic is administered to a subject. In some embodiments, multiple doses of the chemotherapeutic are administered. In some embodiments, the chemotherapeutic is administered at a dose of between 1 mg/kg and 100 mg/kg. In some embodiments, the chemotherapeutic is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. Furthermore, such doses can be administered at multiple times during a day, and/or on multiple days, weeks, or months.

In certain embodiments, the effective dose of a pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### C. Other Agents

It is contemplated that other agents may be used in combination with certain aspects of the present embodiments to improve the therapeutic efficacy of treatment. These additional agents include agents that are useful in the treatment or prevention of cancer. Additional agents also include agents that are useful in the treatment or prevention of pain (e.g., an analgesic such as an opioid, a nonsteroidal anti-inflammatory drug (NSAID), acetaminophen, a steroid, a COX-2 inhibitor, or a topical analgesic).

### IV. Pharmaceutical Compositions

In certain aspects, the disclosed compositions or agents for use in the methods, such as TTI-101, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—TTI-101 is a Competitive Inhibitor of STAT3 that Spares Oxidative Phosphorylation and Reverses Mechanical Allodynia in Mouse Models of Neuropathic Pain

TTI-101 does not affect mitochondrial function.

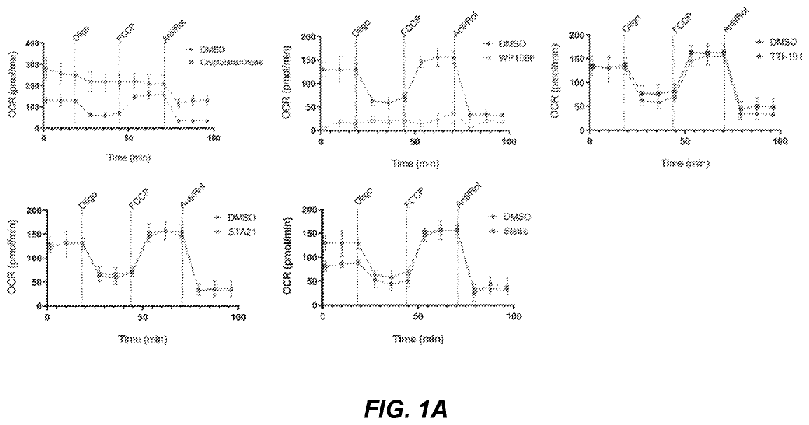

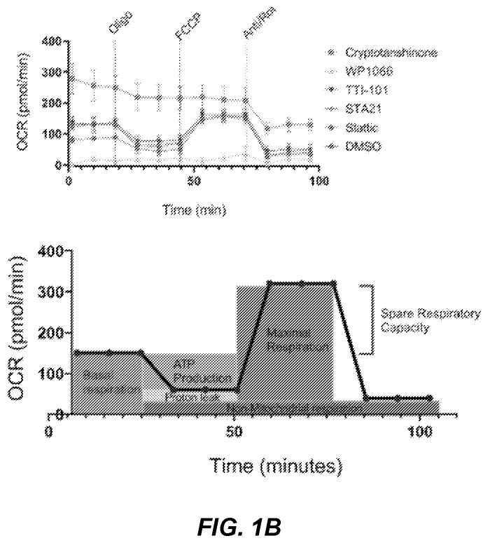

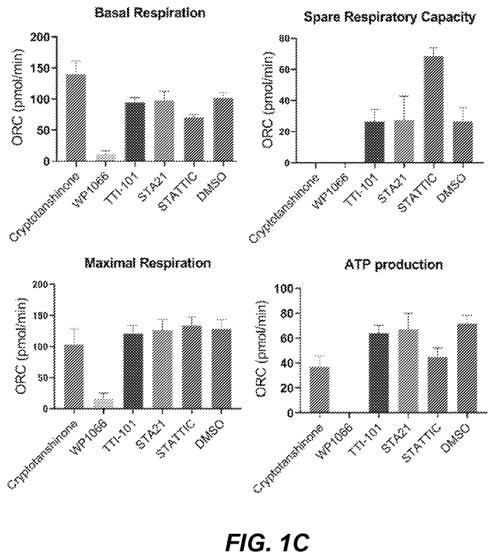

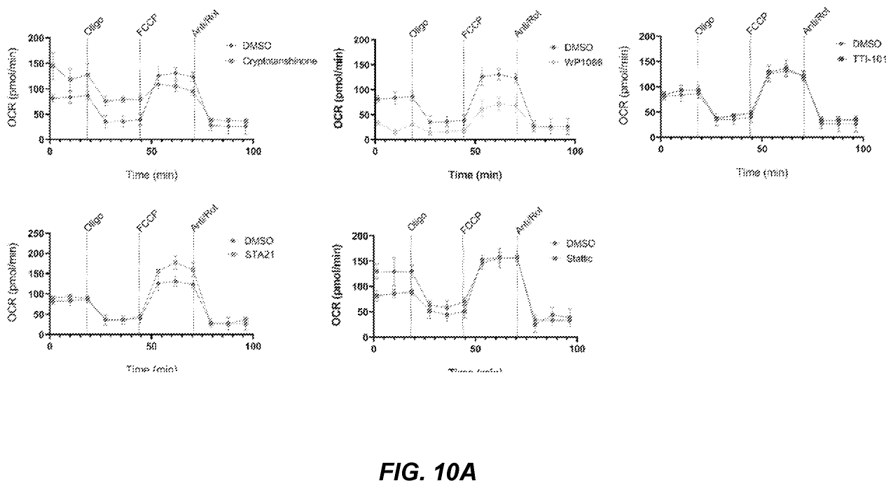

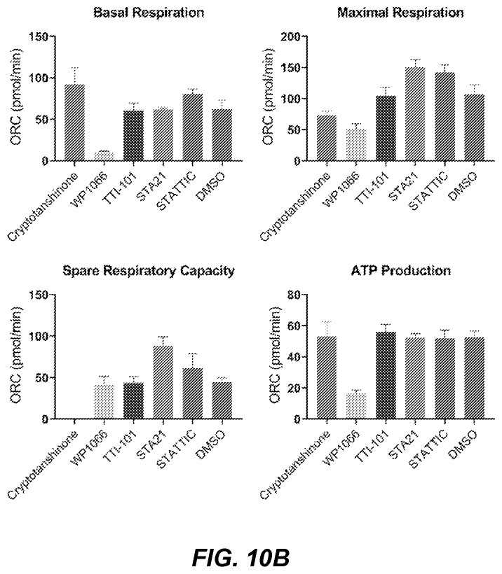

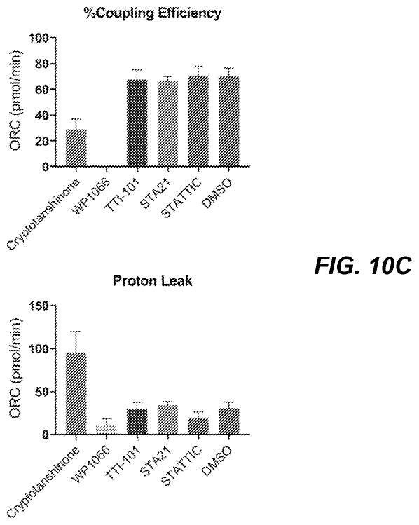

Mitochondrial dysfunction has been demonstrated to contribute to drug-related SEA (Dykens & Will. 2007). Examination of TTI-101 for safety in 28-day IND-enabling studies in rats and dogs (Bharadwaj et al., 2016) did not demonstrate any serious toxicity including lactic acidosis, which is a clinical manifestation of mitochondrial dysfunction. However, to determine if TTI-101 caused subclinical abnormalities in mitochondrial function, the effects of TTI-101 and four other direct STAT3 inhibitors on mitochondrial respiration were examined using a Seahorse XF Cell Mito Stress Test kit that measured basal respiration. ATP production, maximal respiration, proton leak, and spare respiratory capacity. The OCR curves of cells incubated with TTI-101 at concentrations 3-fold and 10-fold higher than its IC₅₀ for STAT3 inhibition (Bharadwaj et al., 2016) were similar to cells treated with DMSO control (FIGS. 1A-1C and FIGS. 10A-10C). Similarly. STA21 and Stattic did not consistently alter the OCR curves compared to DMSO at these concentrations (FIGS. 1A-1C and FIGS. 10A-10C).

In contrast, marked abnormalities were observed in the OCR curves of cells treated with cryptotanshinone and WP1066 (FIGS. 1A-1B). Overall, the effects of cryptotanshinone on the OCR curves mimicked a mitochondrial uncoupler, which creates a ‘shot-circuit’ in the oxidative process by inducing a proton leak (PL) such that the loss of proton motive force proceeds without ATP generation. Cells incubated with cryptotanshinone (10 μM and 30 μM) demonstrated a concentration-dependent increase in basal respiratory rate compared to control cells and decreased responses to oligomycin and to antimycin A/rotenone treatment. In addition, cryptotanshinone at 30 μM concentration blocked cell responses to FCCP treatment and resulted in a 50% reduction in ATP level production. (FIGS. 1A-1C). To further examine the effects of chyrptanshinone on ATP production, the fraction of basal mitochondrial oxygen consumption linked to ATP synthesis (coupling efficiency) was measured; coupling efficiency was significantly reduced (FIG. 10C), further indicative of mitochondrial dysfunction. In addition, the OCR after oligomycin treatment, which is a direct measure of the proton leak rate (FIG. 10C), showed a significant increase in proton leak in cells incubated with cryptotanshinone (30 μM) indicating that mitochondria are uncoupled and severely damaged (Amireddy, Puttapaka, Vinnakota, Ravuri, Thonda & Kalivendi, 2017; Brand & Nicholls, 2011). Similar to cryptotanshinone, marked abnormalities were observed in the OCR curves of cells treated with WP1066 at 10 and 30 μM; (FIGS. 1A-1C and FIGS. 10A-10C) indicative of mitochondrial dysfunction (Brand & Nicholls, 2011), including diminished basal OCR, ATP production rate, maximal respiration, spare respiratory capacity, and coupling efficiency.

TTI-101 does not induce STAT3 aggregation in cells.

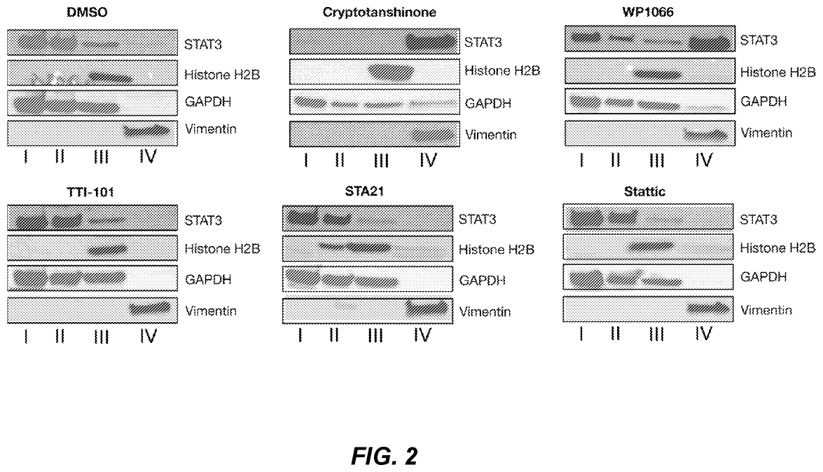

STAT3 inhibitors demonstrated to impair mitochondrial activity also were found to cause STAT3 to aggregate in cells under low glucose conditions (Genini et al., 2017). Using similar experimental conditions, the effects of TTI-101 and other direct STAT3 inhibitors on the partitioning and oligomeric state of STAT3 were assessed. Cells were incubated in medium containing each STAT3 inhibitor at 10 μM final concentration for 16 hours. Cells were fractionated and fractions I through IV were separated by SDS-PAGE and immunoblotted using antibodies selective for each fraction (FIG. 2). TTI-101 had no effect on the intracellular localization of STAT3; similar results were obtained in cells incubated with STA21 or Stattic. In contrast, in cells treated with cryptotanshinone or WP1066, over half of STAT3 was found in the insoluble fraction (Fraction IV) indicating that each induced formation of STAT3 intracellular aggregates, which explains their adverse effects on mitochondrial function.

TTI-101 does not react with GSH or covalently modify STAT3.

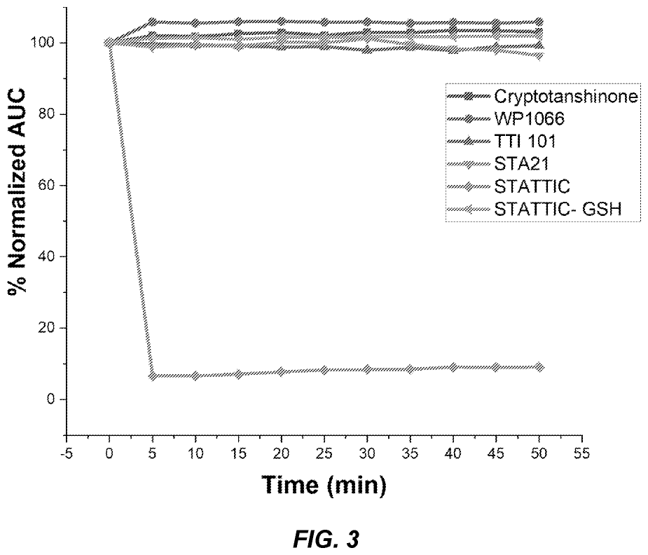

Two studies were performed to directly determine whether TTI-101 mediates its inhibitory effect on STAT3 through covalent modification. The first study was a UV-HPLC-based assay to determine the stability of TTI-101, as well as the other STAT3 inhibitors, in the presence of a natural nucleophile-reduced glutathione (GSH). TTI-101 and the other inhibitors were reconstituted at 100 μM in 50 mM HEPES buffer at pH 7.5 containing 10 mM GSH. Each reaction mixture was sampled at time 0 and every 5 minutes for 50 minutes; all samples were analyzed by HPLC. The amount of unreacted inhibitor was determined by measuring the area under the curve (AUC) and plotting this value as a percentage of the starting AUC as a function of time (FIG. 3). Consistent with early reports of it serving as a Michael's acceptor, Stattic levels decreased rapidly within 5 minutes to <10% of baseline in the presence of GSH while remaining constant in the absence of GSH (FIG. 3). In contrast, there was no loss of TTI-101 in the presence of GSH. 50 minutes after exposure; similar results were observed for cryptotanshinone, WP1066, and STA21.

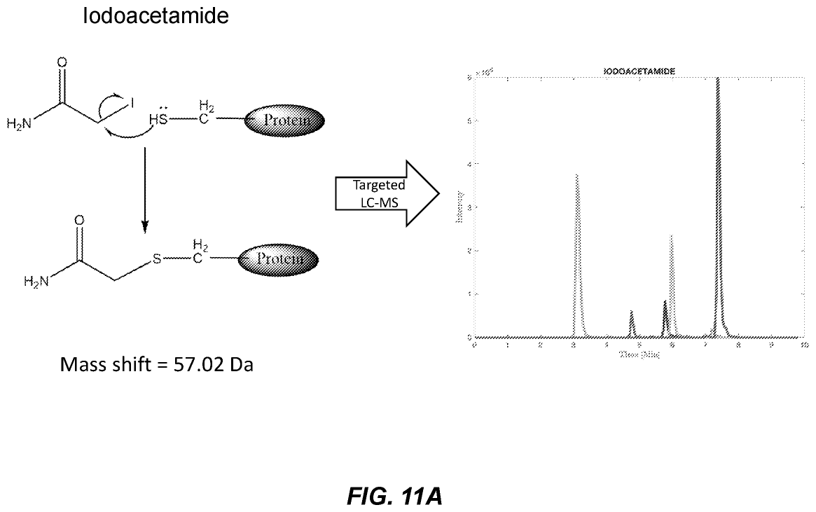

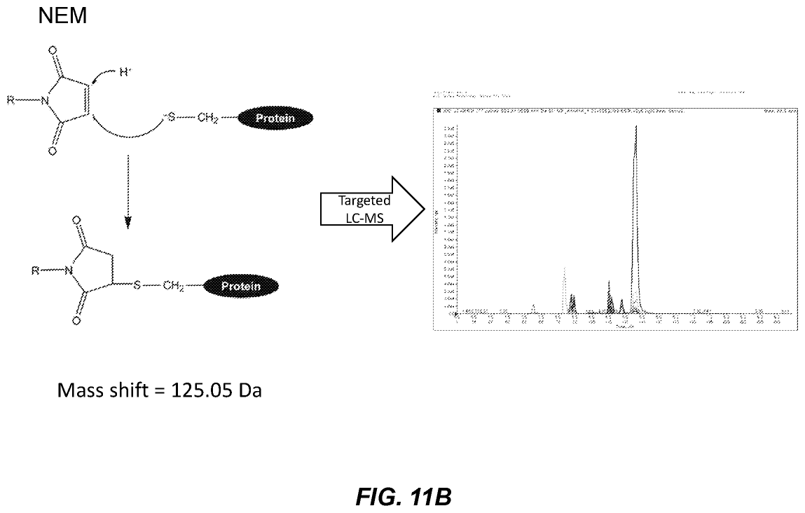

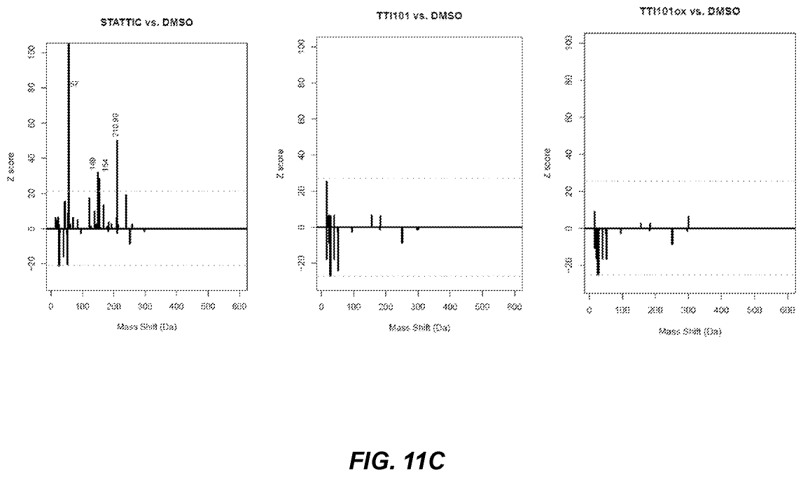

Review of the structure of TTI-101 did not reveal a potential mechanism for alkylation of STAT3 by a Michael addition or by thiol-mediated O-tosyl substitution. However, enol-to-ketone oxidation within the first hydroxy-naphthalene group of TTI-101 forms TTI-101ox, which potentially could undergo a Michael addition reaction. To examine if this was a possibility, recombinant STAT3 was generated post-translationally unmodified in bacteria using a cDNA construct in which the domain containing the N-terminal oligomerization domain was deleted (STAT3βtr); this domain is not necessary for native folding of the core domains of STAT3 (CCD, DBD, linker domain, and SH2 domain) and its removal markedly improves recombinant STAT3 protein solubility. STAT3βtr contains 11 Cys residues. To determine how many of these Cys residues are available to be alkylated when soluble protein is natively folded, we incubated STAT3βtr with two protein alkylating agents, iodoacetamide and N-ethylmaleimide (NEM) under conditions optimal for alkylation (FIG. 11A-11B). Using data obtained on a quadrupole-linear ion trap MS (Sciex QTrap 5500), adducts were detected based on the presence of predicted MRM signal for peptides containing cysteine residues. The identity of the peptides were confirmed by performing full MS/MS spectra on the detected transitions. The LC-MS/MS of the tryptic digested protein revealed 6 peptides alkylated by iodoacetamide and NEM. Five of the peptides contained a single alkylated Cys, while one of the peptides contained two alkylated Cys residues.

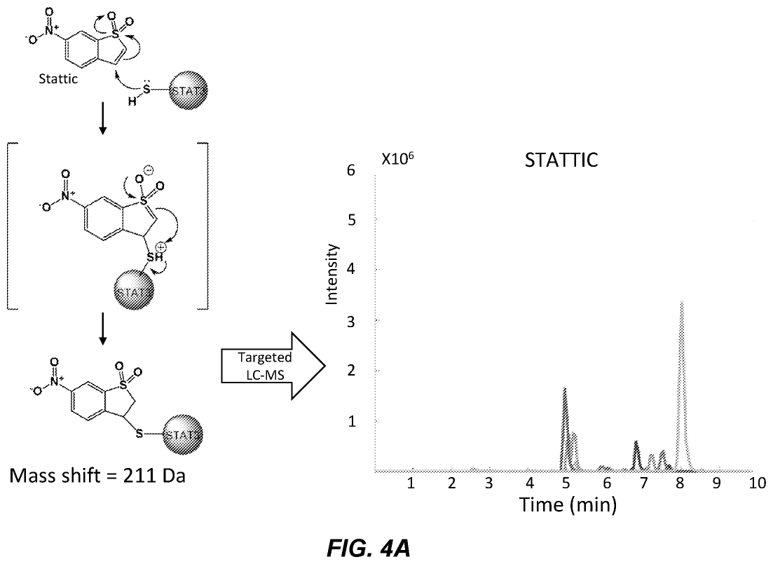

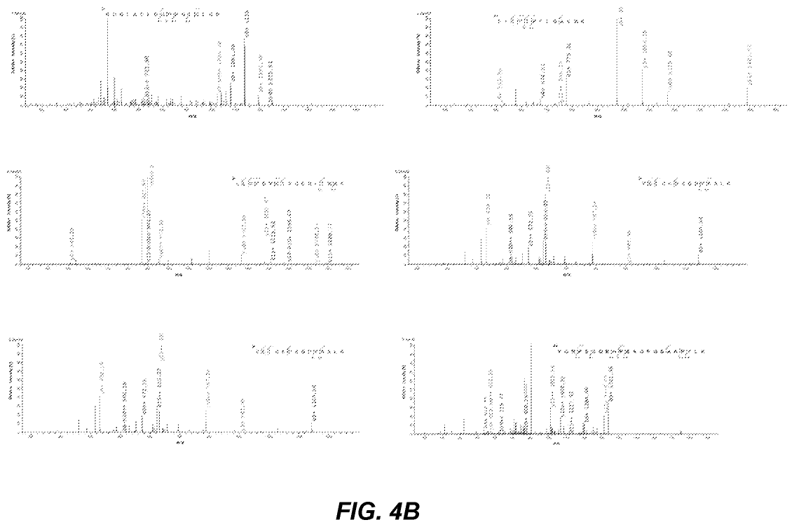

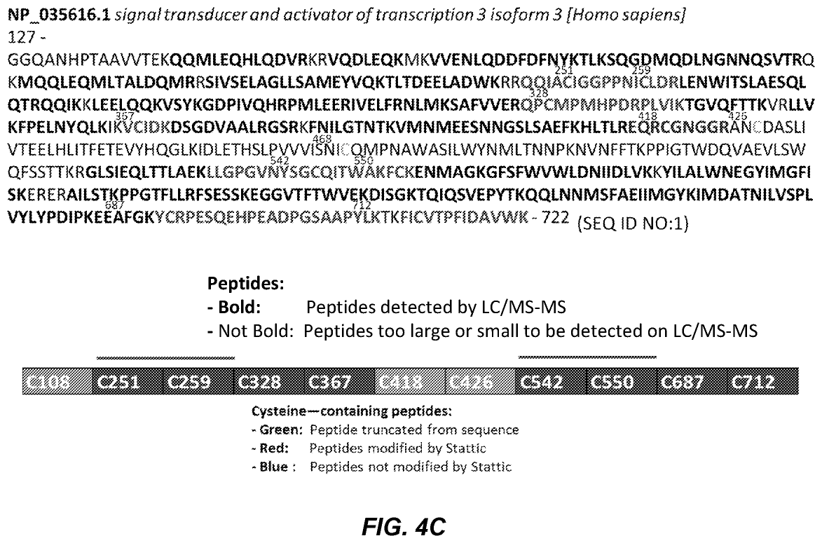

Next, both targeted and untargeted LC-MS/MS analysis was performed on tryptic digests of STAT3βtr incubated under optimal incubation conditions with TTI-101, Stattic, iodoacetamide and NEM: using a Qtrap 5500 and an Orbitrap-Ellite mass spectrometer. The data revealed eight alkylated peptides. (FIGS. 4A-4C)

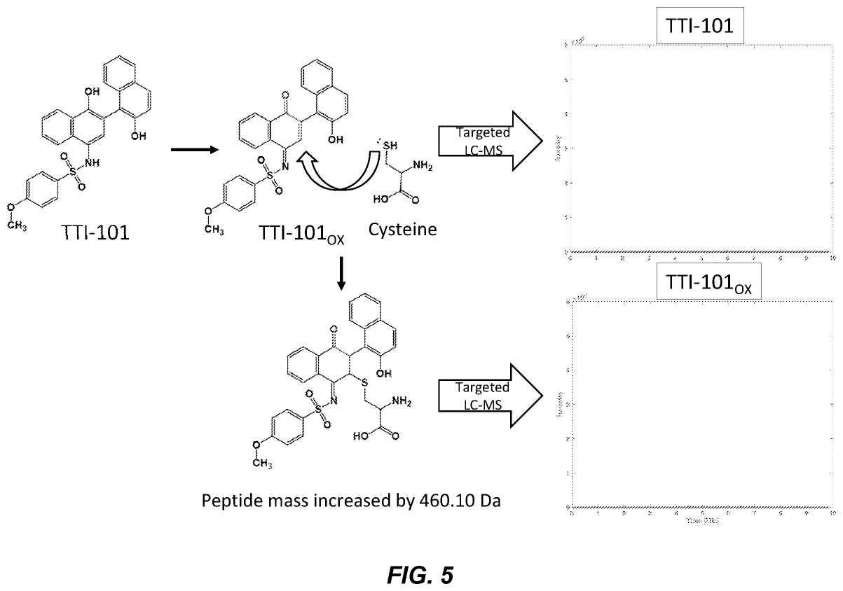

To examine if TTI-101 alkylated STAT3, LC-MS/MS was performed on tryptic digests of STAT3βtr that had been incubated with TTI-101 under native conditions. If TTI-101 alkylated STAT3, one would expect a shift in the mass of peptides containing Cys residues by the equivalent of the exact mass of TTI-101ox, because TTI-101 needs to undergo oxidation at the —OH located para to the sulfonamide group to form a putative Michael's acceptor. No adducts of TTI-101 or TTI-101ox on Cys containing peptides were detected by targeted LC-MS/MS (FIG. 5). To ensure that the failure to detect alkylated protein incubated with TTI-101 was due to insufficient generation of oxidized TTI-101 under the conditions of the reaction, TTI-101ox itself was synthesized and incubated with STAT3βtr. Similar to results obtained with TTI-101, no alkylated peptides were detected upon incubation with TTI-101ox indicating that STAT3 is not alkylated by TTI-101 in either its reduced or oxidized form.

To further support these results, the possibility that TTI-101-reduced or oxidized—may covalently modify STAT3 and result in a mass shift on LC-MS/MS that is not detectable using the targeted detection approach described above was evaluated. High resolution LC-MS/MS analysis of protein digests was performed after incubation of STAT3 with TTI-101 or TTI-101ox using an Orbitrap-Ellite mass spectrometer and analyzed the data using an approach described by Antinori et. al that is tailored for the detection of unknown chemical adduct modifications on proteins (Antinori, Michelot, Lescuyer, Müller & Acosta-Martin, 2019). Using this approach, Stattic adducts were detected in protein digests of STAT3 incubated with Stattic. However, adducts were not identified in digests of STAT3 incubated with either TTI-101 or TTI-101ox confirming that neither forms of TTI-101 covalently modify STAT3 (FIG. 11C).

TTI-101 suppresses chemotherapy-induced mechanical allodynia.

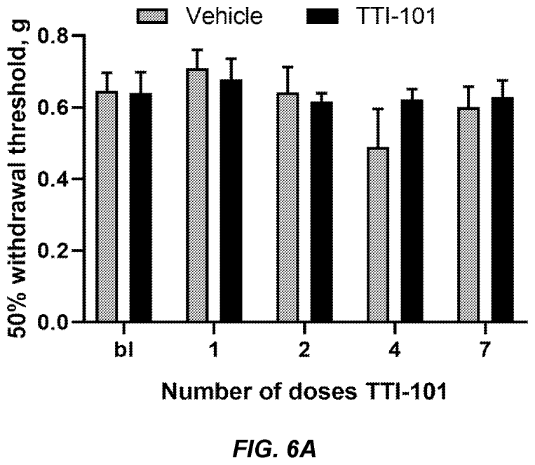

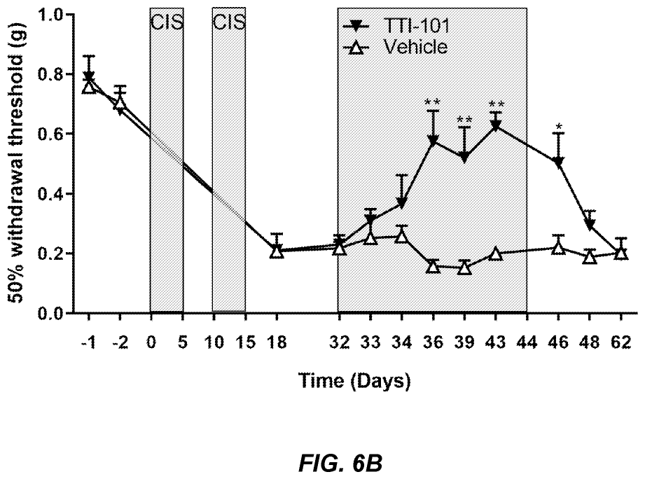

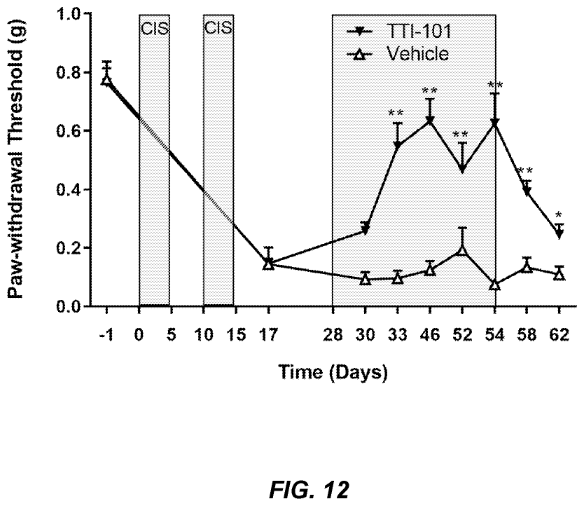

Peripheral neuropathy has been observed with several small-molecule STAT3 inhibitors in clinical-stage development (Bendell et al., 2014; Genini et al., 2017; Ogura et al., 2015). To assess whether TTI-101 causes peripheral neuropathy, male C57BL/6 mice were treated with 7 doses of TTI-101 (50 mg/kg i.p. every other day) and sensitivity to mechanical stimulation was followed over time using von Frey hairs. Administration of TTI-101 alone had no effect on mechanical sensitivity (FIG. 6A). To investigate whether TTI-101 aggravates existing neuropathic pain, the cisplatin model of chemotherapy-induced peripheral neuropathy (CIPN) was used. This model was selected because it was shown previously that it is mediated by mitochondrial damage in the peripheral nervous system (Krukowski et al., 2017; Maj, Ma, Krukowski, Kavelaars & Heijnen, 2017). Mice were treated with two cycles of cisplatin (5 daily doses of 2.3 mg/kg followed by 5 days rest), which induces mechanical allodynia (FIG. 6B) that lasts for at least 75 days (Krukowski et al., 2017). TTI-101 administration (50 mg/kg i.p. every other day for a total of 7 doses) was started 17 days after the last dose of cisplatin, when mechanical allodynia had developed fully. TTI-101 administration markedly reduced cisplatin-induced mechanical allodynia (FIG. 6B). The beneficial effect of TTI-101 developed slowly over time-maximal inhibition was obtained after the 4th dose of TTI-101 and was maintained while dosing continued. Mechanical allodynia returned to levels similar to those in mice treated with cisplatin alone 4 days after the last dose of TTI-101. Similar results were obtained in a separate, independent experiment in which the von Frey test was performed at slightly different timepoints. (FIG. 12).

TTI-101 suppresses SNI-induced mechanical allodynia.

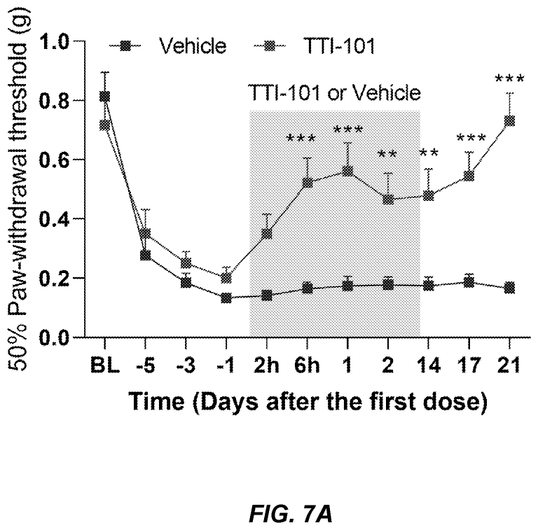

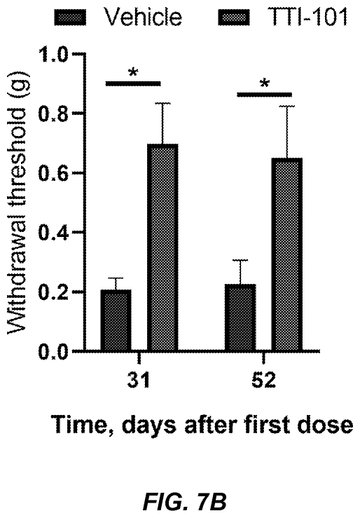

To determine whether the beneficial effects of TTI-101 are limited to CIPN or are more broadly applicable, its effect on mechanical allodynia induced by spared nerve injury (SNI) was examined. SNI induced profound mechanical allodynia in male and female mice. Administration of TTI-101 reduced mechanical allodynia and repeated dosing of TTI-101 led to complete reversal of SNI-induced mechanical allodynia in male and female mice (FIG. 7A). The results in FIG. 7B show that the beneficial effect of TTI-101 was sustained until day 52, which was 40 days after the last dose.

RNA-seq analysis of effect of TTI-101 on the DRG transcriptome in cisplatin-treated mice.

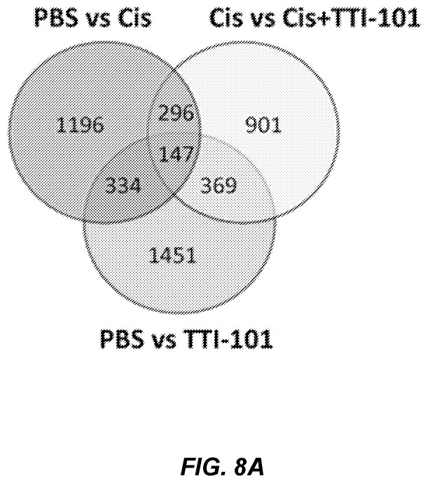

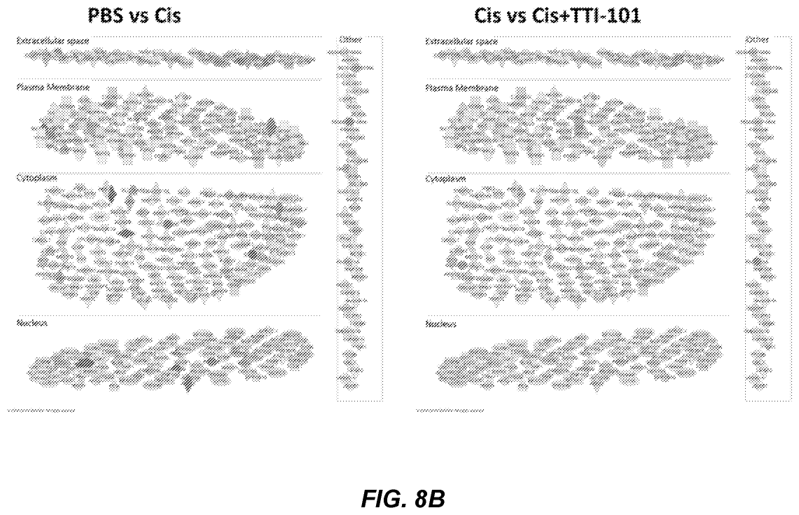

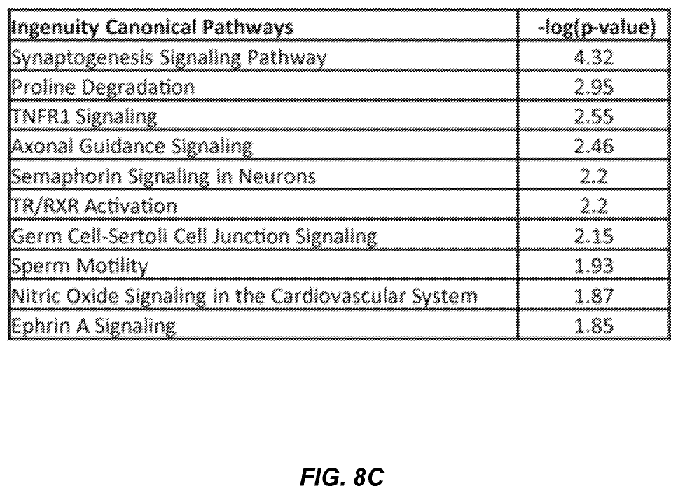

To determine whether the beneficial effect of TTI-101 on CIPN was associated with changes in the transcriptome and, in particular, in expression of STAT3 target genes, RNA-seq analysis was performed on dorsal root ganglia (DRG). Mice were treated with cisplatin followed by TTI-101 as described above and lumbar DRG were collected at 4 hours after the fourth dose of TTI-101 or vehicle. Comparison of the transcriptome in DRG from mice treated with cisplatin vs. PBS showed that cisplatin changed the expression of 1,973 genes (675 down, 1,298 up; FIG. 8A). TTI-101 administration to cisplatin-treated mice changed expression of 1,713 genes (1,416 down, 297 up) vs. mice treated with cisplatin alone. Notably, the 443 genes that were altered in both groups (PBS vs. Cis and Cis vs. Cis+TTI-101) showed an overall opposite expression pattern between groups, indicating that TTI-101 administration normalized the expression of genes whose expression was altered in cisplatin-treated mice (FIG. 8B).

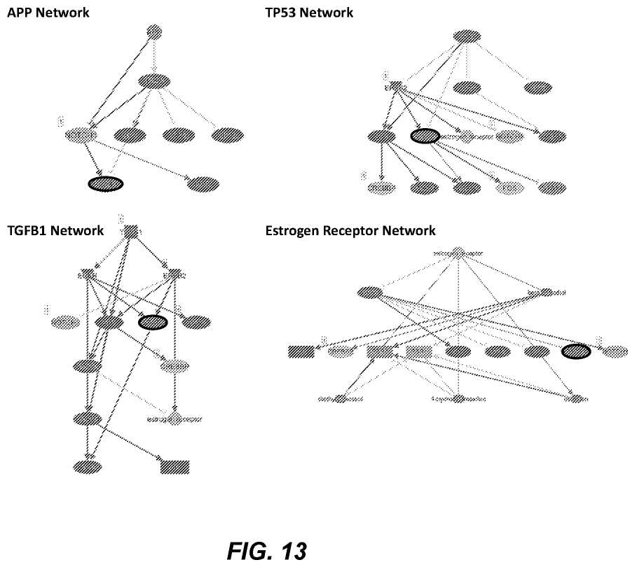

Ingenuity pathway analysis (IPA) focused on canonical pathways (FIG. 8C) and showed that the 443 genes were mainly associated with neuronal health and survival pathways (namely synaptogenesis signaling pathway, TNFR signaling, axonal guidance signaling, Semaphorin Signaling in Neurons, Ephrin signaling). Upstream regulator analysis of the 1,713 differentially expressed genes (DEG) that changed with TTI-101 administration revealed huntingtin (HTT), amyloid beta precursor protein (APP), TP53, TGFβ1, and estrogen receptor as the top five upstream regulators driving downstream changes. The mechanistic network of four regulators identified STAT3 as an intermediate regulator. Consistent with the activity of TTI-101 as a STAT3 inhibitor, STAT3 activity was reduced in all networks (FIG. 13). For example, for the TP53 network, 14 regulators were part of the mechanistic network which together influence expression of 481 genes and out of these 481 genes, 64 target genes are controlled by STAT3.

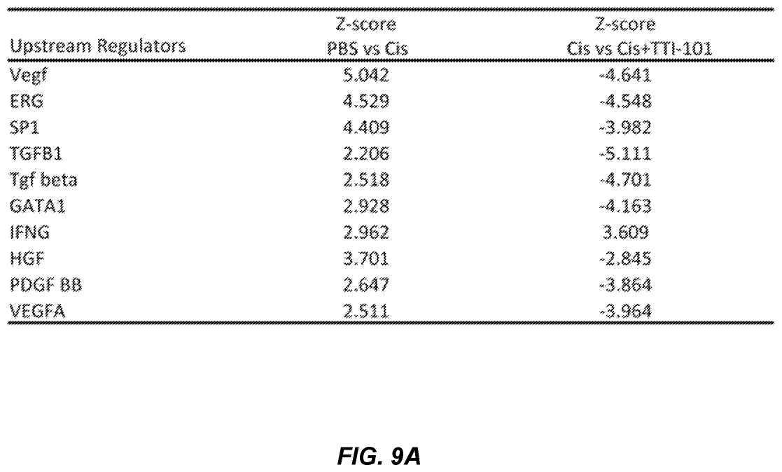

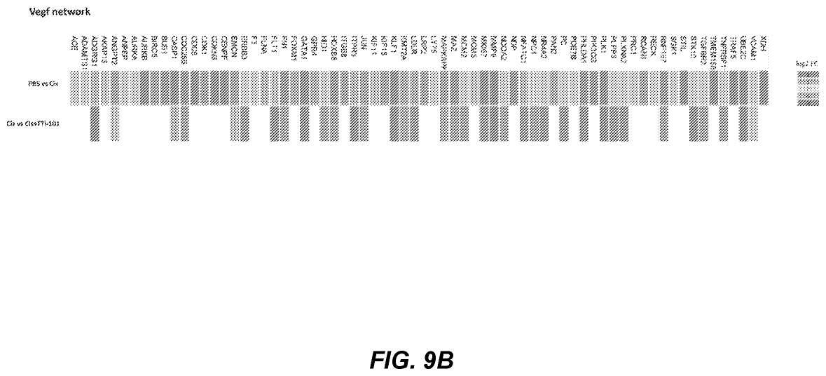

To gain further insight into potential mechanisms underlying TTI-101's reversal of CIPN, IPA was used to perform a comparison analysis (FIG. 9A). IPA identified VEGF as the top upstream regulator different between the groups-PBS vs. Cis and Cis vs. Cis+TTI-101. Specifically, IPA predicted activation of VEGF signaling in mice treated with cisplatin as compared to PBS. This pathway was inhibited when cisplatin-treated mice received TTI-101. The heat map in FIG. 9B shows the effect of TTI-101 administration on the target genes in VEGF signaling network.

### Materials and Methods

Cell lines and chemicals. The human prostate cancer cell line DU-145 was obtained from American Type Culture Collection (ATCC, Rockville, MD, USA) and cultured in RPMI 1640 Medium (ATCC modification) medium containing 10% fetal bovine serum and Antibiotic-Antimycotic (Anti-Anti). The cells were cultured at 37° C. with an atmosphere of 5% CO₂. STAT3 inhibitors-Stattic, chryptotanshinone, WP1066 and STA21 were obtained from Selleck Chemicals (Houston, TX, USA). TTI-101 was custom synthesized by Regis technologies Inc. (Morton Grove, IL, USA. Molecular grade dimethyl sulfoxide (DMSO) was obtained from Sigma-Aldrich (St. Louis, MO, USA). All LC/MS reagents: Ammonium acetate, formic acid, acetonitrile, methanol and water were obtained from Honeywell Fluka (Morris Plains, NJ, USA). STAT3 antibody was purchased from Cell signaling technology (Danvers, MA, USA). Histone H2B (ab52484) and GAPDH (ab9485) antibodies were purchased from Abcam (Toronto, ON, Canada). Antibody to Vimentin (sc66002) was obtained from Santa Cruz biotechnology (Dallas, TX, USA).

Mitochondrial assays. DU-145 cells (2.5×10⁴) were seeded per well in a XF24 plate and incubated at 37° C./5% CO₂ in complete RPMI medium. After 12 Hrs complete medium was replaced with nutrient depleted four-day culture media: conditioned media (CM) and incubated for 4 Hrs. Cells were then treated for another 2 Hrs with STAT3 inhibitors prior to analysis using a Seahorse XF24 Analyzer. The Oxygen Consumption Rate (OCR) was measured in DMEM XF base medium containing 10 mM glucose and 2 mM glutamine and 1 mM pyruvate, before and after the sequential injection of oligomycin, FCCP and rotenone/antimycin A to final concentrations of 1 μM, 1 μM and 0.5 μM respectively. Basal and maximal respiration values were estimate by subtraction of OCR value after treatment of cells with rotenone and antimycin A (which reflects non-mitochondrial respiration) from OCR values in cells treated with oligomycin and FCCP respectively.

Cell fractionation. Cells were treated for 16 hrs with STAT3 inhibitors at a concentration of 10 μM and 1% DMSO in glucose depleted conditioned media (CM), as described (Genini et al., 2017). Lysates were fractionated into cytosol, organelle, nuclei, and cytoskeleton subcellular fractions using ProteoExtract kit (Calbiochem, San Diego, California, United States) according to the manufacturer's directions. Enrichment of each fraction was assessed by SDS-PAGE and immunoblotting using antibodies against GAPDH (cytosol and organelles, Fractions I and II), histone H2B (nucleus, Fraction III), and vimentin (cytoskeleton and insoluble proteins), Fraction IV).

Expression and purification of recombinant STAT3. STAT3 (127-722) cDNA was cloned into a pET15b vector and transformed in BL21 (DE) (Life Technologies, Inc. Woburn, MA) Expression of the recombinant protein was inducing by 0.5 mM IPTG, at 20° C. for 5 hr. The recombinant STAT3 protein was purified by ammonium sulfate precipitation followed by an ion exchange step with HiTrap Q columns (GE Healthcare Bio-Sciences, Uppsala Sweden) and size exclusion chromatography to achieve purity of over 98%.

Glutathione reaction studies. STAT3 inhibitors (10 ul of 10 mM stock in DMSO) were spiked into reaction buffer [50 mM HEPES pH7.5 containing 10 mM reduced glutathione (GSH)] the samples were mixed thoroughly and placed in an autosampler set at 20° C. The reactions were monitored by high performance liquid chromatography (HPLC) using an Exion LC Sciex unit equipped with a UV detector. The stationary phase used was a C18 Synergi™ 4 μm Fusion-RP 80 Å LC column (50×2 mm); the mobile phase was water (A) and acetonitrile (B). The elution process consisted of an isocratic step with 20% mobile phase B, followed by 80% B, for 2 minutes. The flow rate was maintained at 0.5 mL/min during the run. Measurements were conducted at intervals of 5 minutes for a period of 55 minutes total. The presence of the STAT3 inhibitors were quantified by calculating the area under the curves (AUCs) of the compound peaks at 260-295 nm.

STAT3 alkylation studies. Purified recombinant core fragment of STAT3β protein in ammonium bicarbonate buffer (10 μM) was mixed with each compound at a final concentration of 100 μM. The protein mixture was then incubated at 37° C. overnight. Samples were reduced with 5 mM DTT at 37 ºC for one hour and further alkylated with iodoacetamide (15 mM) for 30 minutes at RT in the dark, followed by digestion with trypsin gold in a dry incubator at 37° C. overnight. Formic acid was added the next day to 5% and each protein sample was diluted in 5 mM ammonium acetate containing 0.5% formic acid immediately prior to analysis by Targeted Mass Spectrometry Multiple Reaction Monitoring (MRM).

LC-MS/MS. A QTRAP 5500 Sciex hybrid quadrupole-linear ion trap system with a TurboIonSpray ion source in positive mode and equipped with a Sciex LC Exion liquid chromatography system was used to analyze tryptic digests of STAT3 protein samples treated with STAT3 inhibitors. Fractionation of the samples was done using a Waters Symmetry C18 column (100 Å, 3.5 μm, 4.6 mm×150 mm) and a 30 min linear gradient of acetonitrile with 0.1% formic acid at a flow rate of 300 uL/min. A transition list of cysteine-containing peptides with expected drug-cysteine adducts were generated in Skyline software and exported to the QTRAP mass spectrometer to developed acquisition methods. Resultant raw data files generated in the QTRAP were imported back into Skyline for analysis and additional processing.

High resolution LC/MS. An aliquot of the tryptic digests of STAT3 proteins treated with DMSO (control) and those treated with Stattic and TTI101 were analyzed by LC-MS/MS on an Ultimate 3000 RSLC-Nano chromatograph interfaced to an Orbitrap Fusion high-resolution mass spectrometer (Thermo Scientific, Waltham MA). All MS/MS data were analyzed using Sequest-HT (Thermo Scientific). Proteins were identified by searching the fragment spectra against the Swiss-Prot protein database (EBI). Iodoacetamide derivative of cysteine, stattic adducts of cysteine and the predicted TTI-101 adducts of cysteine were specified as variable modifications. To access for potential unknown modifications, the data was analyzed in MaxQuant using the dependent peptide search option (Tyanova, Temu & Cox, 2016). All peptides output list was analyzed by comparing Stattic, TTI101 and TTI101ox with DMSO treated samples as described in (Antinori, Michelot, Lescuyer, Müller & Acosta-Martin, 2019).

Animals. Male and female C57BL/6J mice were purchased from Jackson Laboratories (Bar Harbor, ME) and housed at The University of Texas MD Anderson Cancer Center animal facility (Houston, TX) on a regular 12-hour light/dark cycle with free access to food and water. Mice were group housed on the same rack in individually ventilated cages. Mice were 8-10 weeks of age at the start of the experiment and were randomly assigned to groups (cage) by animal care givers not involved in the experiment. Investigators were blinded to treatment until group data were analyzed and the code was broken by an investigator not involved in the study. All experimental procedures were consistent with the National Institute of Health Guidelines for the Care and Use of Laboratory Animals and the Ethical Issues of the International Association for the Study of Pain (Zimmermann, 1983) and were approved by the Institution for Animal Care and Use Committee (IACUC) of M.D. Anderson Cancer Center. Experiments were performed and reported in compliance with the ARRIVE guidelines (Kilkenny, Browne, Cuthill, Emerson & Altman, 2010).

Pain measurements and chemotherapy-induced peripheral neuropathy (CIPN). The effect of TTI-101 and chemotherapy on mechanical sensitivity as a read out for pain were assessed over time using von Frey hairs (0.02. 0.07. 0.16. 0.4, 0.6. 1.0, and 1.4 g; Stoelting, (Wood Dale, Illinois, USA) and the up and down method as described previously (Chaplan, Bach, Pogrel, Chung & Yaksh, 1994; Krukowski et al., 2017). Cisplatin (TEVA Pharmaceuticals, North Wales, PA) was diluted in sterile PBS and administered i.p. at a dose of 2.3 mg/kg per day for 5 days followed by 5 days of rest and another 5 days of injections (Mao-Ying et al., 2014).

Spared nerve injury (SNI). SNI surgery was performed on male and female C57BL/6j mice (8 weeks old; Jackson Laboratories), as described (Singhmar et al., 2020). The sural, common peroneal and tibial branches of the sciatic nerve of the left hind paw were exposed under isoflurane anesthesia. A silk suture was used to ligate the common peroneal and tibial branches and 2-4 mm of the distal ends were removed. The sural nerve was left intact. Mice received buprenorphine right before and 1 hour after surgery. Mice were treated with 6 doses of TTI-101 (50 mg/kg in vehicle—60% Labrasol/40% PEG-400—or vehicle alone) administered by oral gavage every other day starting on day 10 after SNI. Mechanical sensitivity was monitored over time using von Frey hairs.

Data and analysis. Studies were designed to include groups of equal size, using randomization and blinded analysis. Statistical analysis was undertaken for studies where each group size was at least n=5, except for the CIPN study. We performed two independent CIPN experiments each with 4 mice per group and slightly different time lines. Animal group size selection for mechanical allodynia was based on previously published data for similar experiments in which sample size calculations were conducted (Singhmar et al., 2020). Pain behavior data were normally distributed and analyzed by Two-way repeated measures ANOVA followed by Tukey post tests using PRISM8 software; P<0.05 was considered statistically significant. Data from all animals enrolled were included in the final analysis. The data and statistical analyses are in accordance with guidelines of the British Journal of Pharmacology on experimental design and analysis in pharmacology (Curtis et al., 2018).

RNA-seq and transcriptome analysis of dorsal root ganglion (DRG). Whole-genome RNA sequencing was used to identify transcriptional changes induced by cisplatin and TTI-101 in the DRG of 3 mice per group. Total RNA was isolated with the RNeasy MinElute Cleanup Kit (Qiagen, Hilden, Germany). Libraries were prepared with the Stranded mRNA-Seq kit (Kapa Biosystems, Wilmington, MA) following the manufacturer's guidelines. Stranded-mRNA seq was performed with a HiSeq4000 Sequencer (Illumina, San Diego, CA) with 76nt PE format by the RNA Sequencing Core at MD Anderson Cancer Center. Data analysis was performed as previously described (Chiu et al., 2018; Ma et al., 2019). Briefly, expression data of three samples per group were analyzed in R using bioconductor packages. STAR was used for alignment of paired-end reads to the mm10 version of the mouse reference genome; feature Counts was used to assign mapped sequence reads to genomic features, and DESeq2 was used to identify differentially expressed genes (padj<0.05). Quality check of raw and aligned reads was performed with FastQC and Qualimap. Next, we used Ingenuity Pathway Analysis (IPA; Qiagen Inc., https://www.qiagenbioinformatics.com/products/ingenuity-pathway-analysis/) for analysis of the canonical pathways implicated by cisplatin-induced transcriptome changes in DRG.

### Example 2—RNA-Seq Analysis of Effect of TTI-101 in Spared Nerve Injury (SNI) Mice

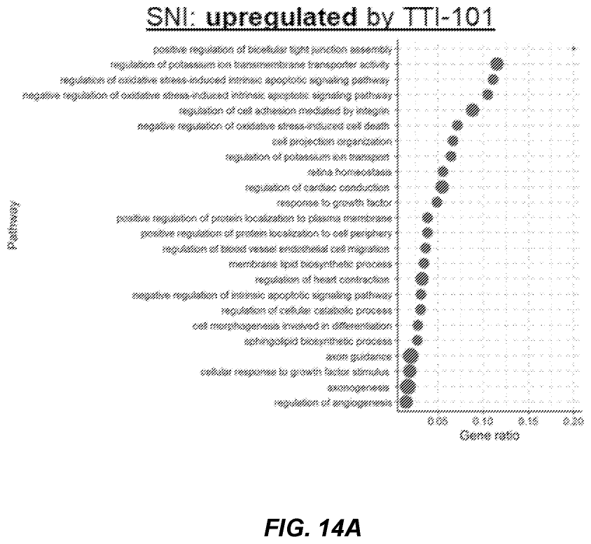

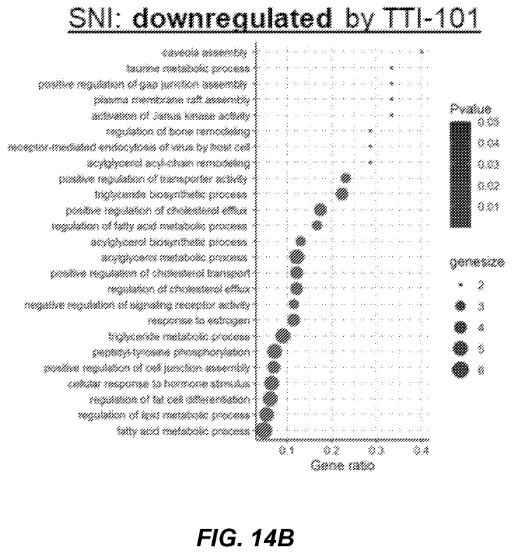

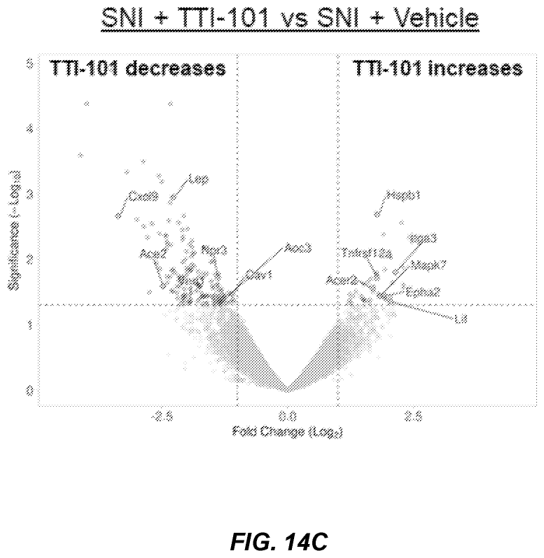

10 days after SNI surgery (performed as described in Example 1), sham and SNI mice were treated with a single dose of vehicle or TTI-101 (50 mg/kg, i.p.). 5 hours later, sciatic nerves were isolated for bulk RNA-sequencing. TTI-101 treatment significantly (adjusted p=<0.05) upregulated 41 genes (FIG. 14A), including those related to cell adhesion and barrier formation (positive regulation of cell adhesion mediated by integrin, positive regulation of gap junction assembly, regulation of bicellular tight junction assembly), reduced oxidative stress (negative regulation of oxidative stress-induced intrinsic apoptotic signaling pathway) and regulation of response to wounding. 148 genes were significantly (p=<0.05) downregulated by TTI-101 (FIG. 14B), including those involved in positive regulation of receptor signaling pathway via JAK-STAT and activation of Janus kinase activity, regulation of cell migration and endothelial cell proliferation, and inflammatory response. Downregulated genes included those associated with inflammation (Aoc3, Cxc19, Vnn1), nitric oxide synthase activity (Npr3), JAK-STAT signaling and positive regulation of protein phosphorylation (Lep) and regulation of gap junction assembly/endothelial cell proliferation (Ace2, Cav1). Upregulated genes included negative regulators of oxidative stress-induced pathways (Mapk7, Hsbp1), integrin-mediated cell adhesion and tight junction assembly (Acer2, Lif, Epha2, Itga3) and regulation of response to wounding (Tnfrsf12a). FIG. 14C shows a volcano plot of significantly downregulated (top left) or upregulated (top right) genes.

These RNA-seq data offer three major insights: First, they identify inflammatory mediator production, oxidative stress and barrier dysfunction as targets of STAT3 signaling. Second, TTI-101 downregulated genes that are known targets of JAK/STAT signaling, further validating the specificity of TTI-101. Third, the inhibition of multiple inflammatory processes while avoiding overt, broad spectrum immunosuppression suggests TTI-101 and STAT3 inhibition can bring about durable changes in neuroinflammation and pain without compromising systemic immune function.

### Example 3—Evaluation of the Effects of TTI-101 In Vivo and In Vitro on Pain Behavior, Barrier Function, Leukocyte Infiltration, and Inflammatory Mediator Production in the Mouse SNI Model of Nerve Injury

SNI surgery is carried out in C57BL/6 mice as previously described [46, 47]. Analgesia is limited to perioperative administration of buprenorphine SR (0.05 mg/kg SQ). Ten days following injury, once pain hypersensitivity has fully developed, animals are randomized to receive TTI-101 (100 mg/kg) in Labrasol/PEG-400 (vehicle) or vehicle alone by oral gavage (OG) daily for 5 consecutive days. Animals then are examined for behavioral pain sensitivity.

Plantar mechanical sensitivity is assessed using hind paw stimulation with von Frey filaments. Frequency of hindlimb withdrawal in response to application of filaments of increasing force is used as a readout of mechanical sensitivity. Plantar cold sensitivity is tested using the plantar cold assay, in which hindpaw withdrawal in response to a cooling source is used as a readout of cold sensitivity. Testing is carried out after habituation at baseline (prior to surgery), then once weekly for 8 weeks following surgery. Daily testing of von Frey and cold sensitivity is carried out during the TTI-101 treatment window. Additional behavioral testing occurs weekly to more extensively validate analgesic efficacy and functional recovery. The mechanical conflict-avoidance (MCA) test uses a chamber of blunt probes to measure aversion to hindpaw mechanical stimuli and serves as an additional assessment of mechanical sensitivity.

Plantar cold sensitivity is measured using the thermal gradient ring assay, which uses a temperature gradient to measure temperature preference/aversion. Finally, paw placement and other gait parameters are assessed using digital gait analysis (Catwalk XT) and a burrowing activity assay (spontaneous clearing of bedding material from a tube). Though abuse liability of TTI-101 has not been reported, conditioned place preference testing is run in a cohort of uninjured mice to detect any potential abuse liability of TTI-101. All experiments are performed by observers blinded to the genotype and drug formulation administered. This additional behavioral testing is done from baseline (8 weeks)—prior to nerve injury—then weekly thereafter until 16 weeks of age.

Mice are euthanized humanely by cardiac puncture and necropsy performed to remove the contralateral and ipsilateral sciatic nerves (SN) and dorsal root ganglia (DRG). This is done at two key time points-three hours after the last dose of TTI-101 on day 5, and 8 weeks post-SNI.

Anticoagulated blood at both time points is used to measure levels of pro- and anti-inflammatory cytokines, including IL-4, IL-6, IL-10, IL-13, TGF-β and IFN-γ by Luminex. One portion of each SN and DRG is snap-frozen and another portion placed in formalin, then paraffin imbedded (FFPE). Cryotome sections of snap-frozen tissues are used to generate protein lysates for measurement of pY-STAT3, IL-1B and IL-6 by Luminex and Western blotting. Cryotome sections of snap-frozen SN and DRG and anticoagulated blood obtained on day 5 is extracted for measurement of levels of TTI-101. Snap-frozen tissues are processed to isolate RNA for RNA sequencing (RNA-seq) analysis.

Microtome sections of FFPE blocks are H&E stained and undergo immunohistochemistry staining and scoring for pY-STAT3, CD68 (macrophages), PGP9.5 (sensory nerves) and GAP43 (regenerating nerves). Detection of oxidative stress in sciatic nerve and DRG via IHC for nitrotyrosine, 4-hydroxynonenal and MnSOD is carried out. Vascular endothelium permeability in nerve is determined by quantifying the extravasation of the fluorescent dye sodium fluorescein. Cross-sections of the nerve are quantified using the EVOS fluorescence microscope, and mean fluorescence intensity and the percent area positive are determined using NIH ImageJ software. In a subset of mice, characterization of SN and DRG macrophage phenotype are analyzed using flow cytometry with conjugated antibodies against CD86, F4/80, CD163 and CD206.

TTI-101 achieves levels in the plasma and tissue adequate to reduce levels of pY-STAT3 in macrophages in SN and DRG, along with a net reduction in blood-nerve barrier permeability and macrophage infiltration of SN and DRG, mechanical and cold hypersensitivity, and antalgic gait patterns/burrowing aversion. TTI-101 shifts RNA-seq gene signatures toward a net promotion of M2-like macrophage polarization, improved tight junction/barrier formation, reduced ROS production, and circulating pro-inflammatory markers.

### Example 4—Evaluation of the Effects of TTI-101 In Vitro on Sensory Neuron Function in the SNI Mouse Model of Nerve Injury

The effects of TTI-101 in vivo are verified in vitro using two complementary live-cell assays. Spontaneous and evoked Ca²⁺ flux in DRG neuron cultures is assessed. Dissociated lumbar L3/L4/L5 DRG neuron properties in uninjured vs injured cells ipsilateral and contralateral to the site of injury are compared, with and without TTI-101 treatment at 5 days post-treatment, and at 8 weeks post-injury. Electrical activity is assessed ex vivo using multi-electrode array recording. Following cell isolation from mice with/without TTI-101 treatment, rates of spontaneous activity in DRG neurons are longitudinally assessed, a readout of hyperexcitability known to contribute to neuropathic pain. Assessment of Ca²⁺ flux allows activity assessment with single cell resolution, which is important in a heterogeneous sensory population. Calcium imaging also serves as a more direct indicator of disruptions to calcium homeostasis i.e. mitochondrial dysfunction, which may result from injury and/or prolonged neuroinflammation. Electrical activity assessment can be carried out longitudinally and is a more proximate indicator of the likely level of sensory input driving neuropathic pain.

TTI-101 treatment reverses elevations in spontaneous activity and baseline cytosolic Ca²⁺ concentration in DRG neurons ipsilateral to the site of injury, indicating a durable normalization of sensory neuron activity.

### Example 5—Evaluation of the Effects of TTI-101 In Vitro on Macrophage-Driven Inflammation and Oxidative Stress

In Order to assess the impact of TTI-101 on STAT3-mediated macrophage inflammation/ROS production, bone marrow-derived macrophages (BMDM) are used and their ability to produce inflammatory mediators and ROS following activation of STAT3 is measured. STAT3 is activated within BMDM in vitro by addition of Ang II (0.1-1 μM for 24 h), or using a cocktail of inflammatory mediators. BMDM are pre-incubated with TTI-101 (0, 1, 3, 10, and 30 μM) for one hour before addition of Ang II or cytokines. The % of F4/80+ macrophages containing elevated levels of the fluorescent ROS-sensitive dye DCFDA is analyzed. The levels of pY-STAT3 in BMDM are determined. The BMDM cytokine expression pattern at the end of the culture period is assessed by intracellular fluorescence for cytosolic IL-10, IL-4, TNF-a and IL-1b.

BMDM produce ROS and proinflammatory cytokines following STAT3 activation, an observation that is paralleled in IHC of injured nerves. TTI-101 treatment reverses this oxidative stress/pro-inflammatory phenotype, which is reflected in the downregulated genes and gene networks identified by RNA-seq.

### Example 6—Evaluation of the Effects of TTI-101 In Vitro and In Vivo on Pain Behavior, Leukocyte Infiltration, and Inflammatory Mediator Production in the Mouse Chronic Constriction Injury (CCI) Model of Nerve Injury

CCI surgery is carried out in mice. Analgesia is limited to perioperative administration of buprenorphine SR (0.05 mg/kg SQ). Ten days following injury, once pain hypersensitivity has fully developed, animals are randomized to receive TTI-101 (100 mg/kg) in Labrasol/PEG-400 (vehicle) or vehicle alone by oral gavage (OG) daily for 5 consecutive days. Animals then are examined for behavioral pain sensitivity. All experiments are performed by observers blinded to the drug formulation administered. This is done from baseline (8 weeks)—prior to nerve injury—then weekly thereafter until 16 weeks of age.

Mice are euthanized humanely by cardiac puncture and necropsy is performed to remove the contralateral and ipsilateral SN and DRG at two time points-three hours after TTI-101 dosing on day 5 and 8 weeks post-CCI, when sustained reversal of pain hyper-sensitivity by TTI-101 is expected. Anticoagulated blood is used to measure levels of pro- and anti-inflammatory cytokines. One portion of each SN and DRG is snap-frozen and another portion placed in formalin, then paraffin imbedded (FFPE). Cryotome sections of the snap-frozen tissue are used to generate protein lysates for measurement of pY-STAT3, IL-1B, and IL-6 by Luminex, and RNA for RNA-seq analysis. In addition, cryotome sections of snap-frozen tissue and anticoagulated blood obtained on day 5 are extracted for measurement of levels of TTI-101. Microtome sections of FFPE blocks are H&E stained and undergo immunohistochemistry staining and scoring for pY-STAT3, CD68 (macrophages), PGP9.5 (sensory nerves) and GAP43 (regenerating nerves). Detection of oxidative stress in sciatic nerve and DRG via IHC for nitrotyrosine or 4-hydroxynonenal adducts and MnSOD is carried out. In a subset of mice, characterization of SN and DRG macrophage phenotype is analyzed using flow cytometry with conjugated antibodies against CD86, F4/80, CD163 and CD206.

TTI-101 achieves levels in the plasma and tissue adequate to reduce levels of pY-STAT3 in macrophages in SN and DRG following CCI, along with a net reduction in macrophage infiltration of the sciatic nerve, mechanical and thermal hypersensitivity, and antalgic gait patterns/burrowing aversion.

### Example 7—Evaluation of the Effects of TTI-101 In Vitro on Sensory Neuron Function in the CCI Mouse Model of Nerve Injury

The effects of TTI-101 in vivo are verified in vitro by assessing spontaneous and evoked Ca²⁺ flux. DRG neuron properties are compared in uninjured vs injured cells ipsi/contra to CCI, with and without TTI-101 treatment at 5 days post-treatment, and at 8 weeks post-surgery. Spontaneous electrical activity is assessed ex vivo using multi-electrode array recording for 3 days in vitro.

TTI-101 treatment reverses the elevated spontaneous activity and baseline cytosolic Ca²⁺ concentration in DRGs ipsilateral to the site of injury, indicating a durable normalization of sensory neuron activity.

### Example 8—Evaluation of the Translational Effects of TTI-101 In Vitro on Pain Behavior, Leukocyte Infiltration, Inflammatory Mediator Production and Healing in a Non-Human Primate SNI Model of Nerve Injury

A total of 12 squirrel monkeys (Saimiri bolivensis; 6 males, 6 females, 4-7 years of age) are habituated and tested for pain sensitivity prior to being subjected to the same SNI traumatic nerve injury model described in rodents. In addition to experimenter-evoked pain quantified in prior non-human primate studies, pain-induced changes in natural behavior (using an ethogram) are assessed, an ethologically-relevant correlate to impaired physical function in patients with chronic pain. Evoked hypersensitivity is performed in animals that have been habituated to manual restraint using standard positive reinforcement training procedures. Mechanical hypersensitivity is determined using the von Frey test. The sensitivity of each foot to a range of von Frey filaments (bending forces: 0.068, 0.17, 0.41, 0.69, 1.5, 3.7, 8.5 and 15 g) are recorded. A single trial consists of presenting each filament to the ventral surface of the foot 5 times in rapid succession (within 2 s). Each trial is repeated 5 times at approximately 15-s intervals. The animals are scored on the number of foot withdrawals during the 5 trials. Cold hypersensitivity is assessed by pipetting drops of acetone on to the ventral surface of each foot. The time spent attending to the foot is recorded in seconds. As a measure of ongoing pain and changes in natural behavior, animals are scored using an ethogram comprised of 41 independent behaviors, including self-care, movement, and abnormal and social behaviors. Focal animal observations of the socially housed study subjects (15-minute) are conducted 5×/week for all subjects throughout the study. Observations are equally distributed across morning, midday, and afternoon time periods. Changes in time spent inactive, interacting socially, and engaged in abnormal behavior are the primary indicators of changes in pain sensitivity.

After habituation to testing procedures is completed (3 weeks), baseline behavioral assessments are carried out in the week prior to surgery. Post-operative analgesia is provided for 7 days post-surgery. Following the post-operative period, 2 additional weeks are included where human standard-of-care (gabapentin) is provided to minimize distress/suffering. Gabapentin is then withdrawn 48 h prior to obtaining post-surgery behavioral readings. Immediately thereafter, animals are randomized to receive TTI-101 (100 mg/kg) in a proprietary self-emulsifying drug delivery (SEDD) vehicle (n=6, 3 males, 3 females), or to receive vehicle alone (n=6, 3 males, 3 females); each is administered by oral gavage (OG) daily for 5 consecutive days.

Blood samples are drawn 3 hours after the last dose of TTI-101 to measure plasma TTI-101 levels using LCMS/MS analysis and for inflammatory mediators identified by RNA-seq and Luminex experiments using a Luminex panel. In order to assess durable changes to the nerve injury microenvironment, animals are necropsied at 1, 2, and 3 weeks after final behavioral assessment (n=2 per time point) and one portion of each SN and DRG is snap-frozen and another portion placed in formalin, then paraffin imbedded (FFPE). Cryotome sections of the snap-frozen tissue are used to generate protein lysates for measurement of pY-STAT3, IL-1B, and IL-6 by Luminex. Microtome sections of FFPE blocks are H&E stained and undergo immunohistochemistry staining and scoring for pY-STAT3, CD68 (macrophages), PGP9.5 (sensory nerves) and GAP43 (regenerating nerves). Detection of oxidative stress in sciatic nerve and DRG via IHC for nitrotyrosine, 4-hydroxynonenal and MnSOD is also carried out.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amireddy N, Puttapaka S N, Vinnakota R L, Ravuri H G, Thonda S, & Kalivendi S V (2017). The unintended mitochondrial uncoupling effects of the FDA-approved anti-helminth drug nitazoxanide mitigates experimental parkinsonism in mice. The Journal of biological chemistry 292: 15731-15743.
Antinori P, Michelot T, Lescuyer P, Müller M, & Acosta-Martin A E (2019). Detection of Unknown Chemical Adduct Modifications on Proteins: From Wet to Dry Laboratory. In Mass Spectrometry of Proteins: Methods and Protocols. eds Evans C. A., Wright P. C., & Noirel J. Springer New York: New York, NY, pp 99-113.
Ball D P, Lewis A M, Williams D, Resetca D, Wilson D J, & Gunning P T (2016). Signal transducer and activator of transcription 3 (STAT3) inhibitor, S31-201, acts as a potent and non-selective alkylating agent. Oncotarget 7: 20669-20679.
Bendell J C, Hong D S, Burris H A, 3rd, Naing A, Jones S F, Falchook G, et al. (2014). Phase 1, open-label, dose-escalation, and pharmacokinetic study of STAT3 inhibitor OPB-31121 in subjects with advanced solid tumors. Cancer Chemother Pharmacol 74: 125-130.
Bennett G J, Doyle T, & Salvemini D (2014). Mitotoxicity in distal symmetrical sensory peripheral neuropathies. Nature reviews Neurology 10: 326-336.
Bharadwaj U, Eckols T K, Xu X, Kasembeli M M, Chen Y, Adachi M, et al. (2016). Small-molecule inhibition of STAT3 in radioresistant head and neck squamous cell carcinoma. Oncotarget 7: 26307-26330.
Bharadwaj U, Kasembeli M M, Robinson P, & Tweardy D J (2020). Targeting Janus Kinases and Signal Transducer and Activator of Transcription 3 to Treat Inflammation, Fibrosis, and Cancer: Rationale, Progress, and Caution. Pharmacol Rev 72: 486-526.
Bharadwaj U, Kasembeli M M, & Tweardy D J (2016). STAT3 Inhibitors in Cancer: A Comprehensive Update. In STAT Inhibitors in Cancer. ed Ward A. C. Springer International Publishing: Cham, pp 95-161.
Brademan D R, Riley N M, Kwiecien N W, & Coon J J (2019). Interactive Peptide Spectral Annotator: A Versatile Web-based Tool for Proteomic Applications. Molecular & cellular proteomics: MCP 18: S193-s201.
Brand M D, & Nicholls D G (2011). Assessing mitochondrial dysfunction in cells. The Biochemical journal 435: 297-312.
Chaplan S R, Bach F W, Pogrel J W, Chung J M, & Yaksh T L (1994). Quantitative assessment of tactile allodynia in the rat paw. Journal of neuroscience methods 53: 55-63.
Chiu G S, Boukelmoune N, Chiang A C A, Peng B, Rao V, Kingsley C, et al. (2018). Nasal administration of mesenchymal stem cells restores cisplatin-induced cognitive impairment and brain damage in mice. Oncotarget 9: 35581-35597.
Colvin L A (2019). Chemotherapy-induced peripheral neuropathy: where are we now? Pain 160 Suppl 1: S1-s10.
Curtis M J, Alexander S, Cirino G, Docherty J R, George C H, Giembycz M A, et al. (2018). Experimental design and analysis and their reporting II: updated and simplified guidance for authors and peer reviewers. British journal of pharmacology 175: 987-993.
Dang C V, Reddy E P, Shokat K M, & Soucek L (2017). Drugging the ‘undruggable’ cancer targets. Nature reviews Cancer 17: 502-508.
Di Cesare Mannelli L, Tenci B, Micheli L, Vona A, Corti F, Zanardelli M, et al. (2018). Adipose-derived stem cells decrease pain in a rat model of oxaliplatin-induced neuropathy: Role of VEGF-A modulation. Neuropharmacology 131: 166-175.
Dominguez E, Rivat C, Pommier B, Mauborgne A, & Pohl M (2008). JAK/STAT3 pathway is activated in spinal cord microglia after peripheral nerve injury and contributes to neuropathic pain development in rat. Journal of neurochemistry 107: 50-60.
Dykens J A, & Will Y (2007). The significance of mitochondrial toxicity testing in drug development. Drug discovery today 12: 777-785.
Garama D J, Harris T J, White C L, Rossello F J, Abdul-Hay M, Gough D J, et al. (2015). A Synthetic Lethal Interaction between Glutathione Synthesis and Mitochondrial Reactive Oxygen Species Provides a Tumor-Specific Vulnerability Dependent on STAT3. Molecular and cellular biology 35: 3646-3656.
Genini D, Brambilla L, Laurini E, Merulla J, Civenni G, Pandit S, et al. (2017). Mitochondrial dysfunction induced by a SH2 domain-targeting STAT3 inhibitor leads to metabolic synthetic lethality in cancer cells. Proceedings of the National Academy of Sciences of the United States of America 114: E4924-e4933.
Giantonio B J, Catalano P J, Meropol N J, O'Dwyer P J, Mitchell E P, Alberts S R, et al. (2007). Bevacizumab in combination with oxaliplatin, fluorouracil, and leucovorin (FOLFOX4) for previously treated metastatic colorectal cancer: results from the Eastern Cooperative Oncology Group Study E3200. Journal of clinical oncology: official journal of the American Society of Clinical Oncology 25: 1539-1544.
Kasembeli M M, Bharadwaj U, Robinson P, & Tweardy D J (2018). Contribution of STAT3 to Inflammatory and Fibrotic Diseases and Prospects for its Targeting for Treatment. International journal of molecular sciences 19.
Kilkenny C, Browne W J, Cuthill I C, Emerson M, & Altman D G (2010). Improving bioscience research reporting: the ARRIVE guidelines for reporting animal research. PLOS biology 8: e1000412.
Krukowski K, Ma J, Golonzhka O, Laumet G O, Gutti T, van Duzer J H, et al. (2017). HDAC6 inhibition effectively reverses chemotherapy-induced peripheral neuropathy. Pain 158: 1126-1137.
Li Y Y, Li H, Liu Z L, Li Q, Qiu H W, Zeng L J, et al. (2017). Activation of STAT3-mediated CXCL12 up-regulation in the dorsal root ganglion contributes to oxaliplatin-induced chronic pain. Molecular pain 13: 1744806917747425.
Liu S, Mi W L, Li Q, Zhang M T, Han P, Hu S, et al. (2015). Spinal IL-33/ST2 Signaling Contributes to Neuropathic Pain via Neuronal CaMKII-CREB and Astroglial JAK2-STAT3 Cascades in Mice. Anesthesiology 123: 1154-1169.
Ma J, Kavelaars A, Dougherty P M, & Heijnen C J (2018). Beyond symptomatic relief for chemotherapy-induced peripheral neuropathy: Targeting the source. Cancer 124: 2289-2298.
Ma J, Trinh R T, Mahant I D, Peng B, Matthias P, Heijnen C J, et al. (2019). Cell-specific role of histone deacetylase 6 in chemotherapy-induced mechanical allodynia and loss of intraepidermal nerve fibers. Pain 160: 2877-2890.
Maj M A, Ma J, Krukowski K N, Kavelaars A, & Heijnen C J (2017). Inhibition of Mitochondrial p53 Accumulation by PFT-μ Prevents Cisplatin-Induced Peripheral Neuropathy. Frontiers in molecular neuroscience 10: 108.
Mao-Ying Q L, Kavelaars A, Krukowski K, Huo X J, Zhou W, Price T J, et al. (2014). The anti-diabetic drug metformin protects against chemotherapy-induced peripheral neuropathy in a mouse model. PloS one 9: e100701.
McMurray J S (2006). A new small-molecule Stat3 inhibitor. Chem Biol 13: 1123-1124. Oral STAT3 Inhibitor, TTI-101, in Patients With Advanced Cancers. [Online] Available on the world wide web at ClinicalTrials.gov/show/NCT03195699. [Accessed].
Niu G, Wright K L, Huang M, Song L, Haura E, Turkson J, et al. (2002). Constitutive Stat3 activity up-regulates VEGF expression and tumor angiogenesis. Oncogene 21: 2000-2008.
Ogura M, Uchida T, Terui Y, Hayakawa F, Kobayashi Y, Taniwaki M, et al. (2015). Phase I study of OPB-51602, an oral inhibitor of signal transducer and activator of transcription 3, in patients with relapsed/refractory hematological malignancies. Cancer Sci 106: 896-901.
Selvaraj D, Gangadharan V, Michalski C W, Kurejova M, Stösser S, Srivastava K, et al. (2015). A Functional Role for VEGFR1 Expressed in Peripheral Sensory Neurons in Cancer Pain. Cancer cell 27: 780-796.
Singhmar P, Trinh R T P, Ma J, Huo X, Peng B, Heijnen C J, et al. (2020). The fibroblast-derived protein PI16 controls neuropathic pain. Proceedings of the National Academy of Sciences of the United States of America 117: 5463-5471.
Sun Y, Ju M, Lin Z, Fredrick T W, Evans L P, Tian K T, et al. (2015). SOCS3 in retinal neurons and glial cells suppresses VEGF signaling to prevent pathological neovascular growth. Science signaling 8: ra94.
Tyanova S, Temu T, & Cox J (2016). The MaxQuant computational platform for mass spectrometry-based shotgun proteomics. Nature protocols 11: 2301-2319.
Verheyen A, Peeraer E, Nuydens R, Dhondt J, Poesen K, Pintelon I, et al. (2012). Systemic anti-vascular endothelial growth factor therapies induce a painful sensory neuropathy. Brain: a journal of neurology 135: 2629-2641.
Ward J A, Pinto-Fernandez A, Cornelissen L, Bonham S, Diaz-Saez L, Riant O, et al. (2020). Re-Evaluating the Mechanism of Action of alpha, beta-Unsaturated Carbonyl DUB Inhibitors b-AP15 and VLX1570: A Paradigmatic Example of Unspecific Protein Cross-linking with Michael Acceptor Motif-Containing Drugs. J Med Chem.
Wei D, Le X, Zheng L, Wang L, Frey J A, Gao A C, et al. (2003). Stat3 activation regulates the expression of vascular endothelial growth factor and human pancreatic cancer angiogenesis and metastasis. Oncogene 22: 319-329.
Xu T, Zhang X L, Ou-Yang H D, Li Z Y, Liu C C, Huang Z Z, et al. (2017). Epigenetic upregulation of CXCL12 expression mediates antitubulin chemotherapeutics-induced neuropathic pain. Pain 158: 637-648.
Xu X, Kasembeli M M, Jiang X, Tweardy B J, & Tweardy D J (2009). Chemical probes that competitively and selectively inhibit Stat3 activation. PloS one 4: e4783.
Yang R, & Rincon M (2016). Mitochondrial Stat3, the Need for Design Thinking. International journal of biological sciences 12: 532-544.
Zimmermann M (1983). Ethical guidelines for investigations of experimental pain in conscious animals. Pain 16: 109-110.

What is claimed is:

1. A method of treating a subject for mechanical nerve injury, the method comprising administering to the subject a therapeutically effective amount of a pharmaceutical composition comprising TTI-101 or a pharmaceutically acceptable salt thereof.

2. The method of claim 1, wherein the pharmaceutical composition is administered to the subject orally.

3. The method of claim 1 or 2, wherein the mechanical nerve injury is traumatic nerve injury.

4. The method of claim 1 or 2, wherein the mechanical nerve injury is carpal tunnel syndrome.

5. The method of claim 1 or 2, wherein the mechanical nerve injury is vertebral disk herniation.

6. The method of any of claims 1-5, wherein administering the pharmaceutical composition to the subject completely alleviates one or more symptoms of the mechanical nerve injury in the subject.

7. The method of claim 6, wherein the one or more symptoms comprise allodynia.

8. The method of claim 6 or 7, wherein the one or more symptoms comprise hyperalgesia.

9. The method of any of claims 6-8, wherein the pharmaceutical composition is not administered to the subject for at least 50 days following complete alleviation of the one or more symptoms.

10. The method of any of claims 1-9, wherein the subject does not have cancer.

11. The method of any of claims 1-9, wherein the subject is not suspected of having cancer.

12. The method of any of claims 1-11, wherein mitochondrial function of the subject following administration of the pharmaceutical composition is not reduced relative to mitochondrial function of the subject prior to administration of the pharmaceutical composition.

13. The method of any of claims 1-12, further comprising administering to the subject an additional therapeutic agent.

14. The method of claim 13, wherein the additional therapeutic agent is an analgesic.

15. The method of claim 14, wherein the analgesic is an opioid, a nonsteroidal anti-inflammatory drug (NSAID), acetaminophen, a steroid, a COX-2 inhibitor, a topical analgesic, or any combination thereof.

16. The method of any of claims 1-15, wherein the subject was previously treated for the mechanical nerve injury with a previous treatment.

17. The method of claim 16, wherein the subject was determined to be resistant to the previous treatment.

18. The method of any of claims 1-17, wherein the TTI-101 or pharmaceutically acceptable salt thereof is administered at a concentration of at most 15 mg/kg.

19. The method of any of claims 1-17, wherein the TTI-101 or pharmaceutically acceptable salt thereof is administered at a concentration of at most 5 mg/kg.

20. The method of any of claims 1-17, wherein the TTI-101 or pharmaceutically acceptable salt thereof is administered at a concentration of at most 1 mg/kg.

21. The method of any of claims 1-20, wherein the pharmaceutical composition is administered to the subject once per day for multiple days.

22. The method of any of claims 1-20, wherein the pharmaceutical composition is administered to the subject every other day for multiple days.

23. A method of treating a subject for chemotherapy-induced peripheral neuropathy, the method comprising administering to the subject a therapeutically effective amount of a pharmaceutical composition comprising TTI-101 or a pharmaceutically acceptable salt thereof.

24. The method of claim 23, wherein the pharmaceutical composition is administered to the subject orally.

25. The method of any of claims 23-24, wherein mitochondrial function of the subject following administration of the pharmaceutical composition is not reduced relative to mitochondrial function of the subject prior to administration of the pharmaceutical composition.

26. The method of any of claims 23-25, wherein the subject was previously treated with a chemotherapeutic.

27. The method of claim 26, wherein the chemotherapeutic is a platinum-containing chemotherapeutic.

28. The method of claim 27, wherein the chemotherapeutic is cisplatin.

29. The method of claim 27, wherein the chemotherapeutic is oxaliplatin.

30. The method of claim 27, wherein the chemotherapeutic is carboplatin.

31. The method of any of claims 23-25, further comprising administering to the subject a chemotherapeutic.

32. The method of claim 31, wherein the pharmaceutical composition comprises the chemotherapeutic.

33. The method of claim 31, wherein the pharmaceutical composition does not comprise the chemotherapeutic.

34. The method of any of claims 31-33, wherein the pharmaceutical composition and the chemotherapeutic are administered substantially simultaneously.

35. The method of any of claims 31-33, wherein the pharmaceutical composition and the chemotherapeutic are administered sequentially.

36. The method of claim 35, wherein the pharmaceutical composition is administered prior to administering the chemotherapeutic.

37. The method of claim 35, wherein the pharmaceutical composition is administered subsequent to administering the chemotherapeutic.

38. The method of any of claims 31-37, wherein the chemotherapeutic is a platinum-containing chemotherapeutic.

39. The method of claim 38, wherein the chemotherapeutic is cisplatin.

40. The method of claim 38, wherein the chemotherapeutic is oxaliplatin.

41. The method of claim 38, wherein the chemotherapeutic is carboplatin.

42. The method of any of claims 23-41, wherein the subject was previously treated for chemotherapy-induced peripheral neuropathy with a previous treatment.

43. The method of claim 42, wherein the subject was determined to be resistant to the previous treatment.

44. The method of any of claims 23-43, wherein the TTI-101 or pharmaceutically acceptable salt thereof is administered at a concentration of at most 15 mg/kg.

45. The method of any of claims 23-43, wherein the TTI-101 or pharmaceutically acceptable salt thereof is administered at a concentration of at most 5 mg/kg.

46. The method of any of claims 23-43, wherein the TTI-101 or pharmaceutically acceptable salt thereof is administered at a concentration of at most 1 mg/kg.

47. The method of any of claims 23-46, wherein the pharmaceutical composition is administered to the subject once per day for multiple days.

48. The method of any of claims 23-47, wherein the pharmaceutical composition is administered to the subject every other day for multiple days.

49. A method of treating a subject for a neuropathic pain condition, the method comprising administering to the subject a therapeutically effective amount of a pharmaceutical composition comprising TTI-101 or a pharmaceutically acceptable salt thereof.

50. The method of claim 49, wherein the pharmaceutical composition is administered to the subject orally.

51. The method of claim 49 or 50, wherein the neuropathic pain condition is mechanical nerve injury, a metabolic disease, a neurotropic viral disease, an inflammatory condition, nervous system focal ischemia, or a combination thereof.

52. The method of claim 51, wherein the neuropathic pain condition is mechanical nerve injury.

53. The method of claim 52, wherein the mechanical nerve injury is traumatic nerve injury.

54. The method of claim 52, wherein the mechanical nerve injury is carpal tunnel syndrome.

55. The method of claim 52, wherein the mechanical nerve injury is vertebral disk herniation.

56. The method of any of claims 49-55, wherein the administering the pharmaceutical composition to the subject completely alleviates one or more symptoms of the neuropathic pain condition in the subject.

57. The method of claim 56, wherein the one or more symptoms are allodynia.

58. The method of claim 56, wherein the one or more symptoms are hyperalgesia.

59. The method of any of claims 56-58, wherein the pharmaceutical composition is not administered to the subject for at least 50 days following complete alleviation of the one or more symptoms.

60. The method of any of claims 49-59, wherein mitochondrial function of the subject following administration of the pharmaceutical composition is not reduced relative to mitochondrial function of the subject prior to administration of the pharmaceutical composition.

61. The method of any of claims 49-60, wherein the subject does not have cancer.

62. The method of any of claims 49-60, wherein the subject is not suspected of having cancer.

63. The method of any of claims 49-62, further comprising administering to the subject an additional therapeutic agent.

64. The method of claim 63, wherein the additional therapeutic agent is an analgesic.

65. The method of claim 64, wherein the analgesic is an opioid, a nonsteroidal anti-inflammatory drug (NSAID), acetaminophen, a steroid, a COX-2 inhibitor, a topical analgesic, or any combination thereof.

66. The method of any of claims 49-65, wherein the subject was previously treated for the neuropathic pain condition with a previous treatment.

67. The method of claim 66, wherein the subject was determined to be resistant to the previous treatment.

68. The method of any of claims 49-67, wherein the TTI-101 or pharmaceutically acceptable salt thereof is administered at a concentration of at most 15 mg/kg.

69. The method of any of claims 49-67, wherein the TTI-101 or pharmaceutically acceptable salt thereof is administered at a concentration of at most 5 mg/kg.

70. The method of any of claims 49-67, wherein the TTI-101 or pharmaceutically acceptable salt thereof is administered at a concentration of at most 1 mg/kg.

71. The method of any of claims 49-70, wherein the pharmaceutical composition is administered to the subject once per day for multiple days.

72. The method of any of claims 49-70, wherein the pharmaceutical composition is administered to the subject every other day for multiple days.

73. A method of completely alleviating a symptom of traumatic nerve injury in a subject, the method comprising orally administering to the subject a therapeutically effective amount of a pharmaceutical composition comprising TTI-101 or a pharmaceutically acceptable salt thereof.

74. The method of claim 73, wherein the symptom is allodynia.

75. The method of claim 73, wherein the symptom is hyperalgesia.

76. A method of completely alleviating a symptom of chemotherapy-induced peripheral neuropathy in a subject, the method comprising orally administering to the subject a therapeutically effective amount of a pharmaceutical composition comprising TTI-101 or a pharmaceutically acceptable salt thereof.

77. The method of claim 76, wherein the symptom is allodynia.

78. The method of claim 76, wherein the symptom is hyperalgesia.
